SEQUENCE LISTING (49 sequences)

SEQ ID NO: 1 - protein, 72 aa, synthetic construct
MTTIYQIEHYSMDDLASLKIPEQFRHAIWKGILDHRQLHEFSSPSHLLRTPSSASTVSVGSSETRGERVIDA

SEQ ID NO: 2 - DNA, 219 bp, synthetic construct
  source 1..219: mol_type = other DNA
atgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgcttaa

SEQ ID NO: 3 - DNA, 660 bp, synthetic construct
  source 1..660: mol_type = other DNA
atgtctctcgtgggcggatttcctcaccaccctgtggtgcaccatgagggctatccttttgctgccgctgccgcagccgccgctgctgctgcagctagtagatgtagccacgaggaaaacccctacttccacggctggctgatcggccaccctgagatgagccctccagattacagcatggccctgagctacagccctgagtatgcttctggagccgctggactggatcactctcattatggcggagtgcctccaggcgctggacctcctggactgggaggacctagacctgtgaagagaagaggcaccgccaaccggaaagagcggagaagaacccagagcatcaatagcgccttcgccgagctgagagaatgcatccctaatgtgcccgccgacaccaagctgagcaagatcaaaaccctgcggctggccaccagctatatcgcctatctgatggacctgctggccaaggacgatcagaatggcgaggccgaggccttcaaggccgagatcaagaaaaccgacgtgaaagaggaaaagcgcaagaaagagctgaacgagatcctgaagtccaccgtgtccagcaacgacaaaaagaccaagggcagaaccggctggcctcagcatgtgtgggctctggaactgaaacagggcagcggc

SEQ ID NO: 4 - DNA, 2961 bp, synthetic construct
  source 1..2961: mol_type = other DNA
atgaccctgctgggcagcgagcacagcctgctgatcagatccaagttcagaagcgtgctgcagctgagactgcagcagagaagaacacaggaacagctggccaaccagggcatcatccccccactgaaaagacctgccgagttccacgagcagagaaagcacctggacagcgacaaggccaagaacagcctgaagcggaaggcccggaatagatgcaatagcgccgacctggtgaacatgcacatcctgcaggcttccaccgccgagagatctatccctacagcccagatgaagctgaagagagccagactggccgacgacctgaatgagaagattgccctgaggcctggccccctggaactggtggaaaagaacatcctgcctgtggacagcgccgtgaaagaggccatcaagggcaatcaggtgtccttcagcaagagcaccgacgccttcgccttcgaggaagattctagctctgacggcctgtctcctgatcagaccagatctgaagatcctcagaatagcgccggcagccctcctgatgccaaagcctctgatacaccttctaccggcagcctgggcaccaatcaggatctggcctctggcagcgagaacgacagaaatgatagcgccagccagcctagccaccagtctgatgctggaaaacagggcctgggccctccttctacacctattgctgtgcacgccgccgtgaagtctaagagcctgggcgacagcaagaaccggcacaagaagcctaaggaccccaagcccaaagtgaagaagctgaagtaccaccagtacatcccccccgaccagaaggccgaaaagtcccctcctcctatggattccgcctacgctagactgctgcaacagcagcagctgttcctgcagctccagatcctgtctcaacaacagcaacagcagcagcaccggttcagctatctgggaatgcaccaggcccagctgaaagaacccaatgagcagatggtccgcaaccccaatagcagctccacccctctgagcaatacccccctgagccctgtgaagaatagcttttctggccagaccggcgtgtccagctttaagcctggacctctgccccccaacctggacgatctgaaagtgtctgaactgcggcagcagctgagaatcagaggactgcctgtgtctggcaccaagaccgccctgatggatagactgaggccctttcaggactgcagcggcaaccctgtgcccaactttggcgatatcaccaccgtgaccttccccgtgacacccaacaccctgcctaattaccagagcagcagctctaccagcgccctgagcaatggcttctaccactttggcagcacaagcagcagccctccaatcagccctgcctcttctgatctgtctgtggccggaagcctgcccgacacctttaatgatgccagccctagctttggcctgcacccttctccagtgcacgtgtgcacagaggaatccctgatgtctagcctgaatggcggctctgtgccttctgagctggatggcctggattccgagaaggacaagatgctggtggaaaaacagaaagtgatcaacgagctgacctggaagctgcagcaggaacagagacaggtggaagaactgcggatgcagctgcagaagcagaagcggaacaactgctccgagaagaagcctctgccattcctggccgccagcattaagcaggaagaggccgtgtcaagctgcccattcgccagtcaggtgccagtgaagagacagagcagctcctctgaatgtcaccctcctgcttgtgaagctgcccagctgcagcctctgggaaatgcccattgtgtggaaagcagcgaccagaccaatgtgctgagcagcaccttcctgagccctcagtgttctcctcagcattctcccctgggcgctgtgaagtctccacagcacatttctctgccccctagccccaacaaccctcactttctgccatctagttctggcgcccagggcgagggacatagagtgtctagtcctatcagcagccaggtctgcaccgctcagaactctggcgctcatgatggccaccctccaagctttagccctcactcttctagcctgcacccaccttttagcggagcccaggctgattcttctcatggcgctggcggcaatccttgccctaagtctccttgcgtgcagcagaaaatggccggcctgcacagctctgacaaagtgggccctaagttcagcatccccagccccacctttagcaagtctagcagcgccatcagcgaagtgacccagcctccatcttacgaggatgccgtgaagcagcagatgaccagatcccagcagatggacgagctgctggatgtgctgatcgagtctggcgaaatgcctgccgatgccagagaggatcacagctgtctgcagaaggtgcccaagatccccagaagctccagatctcctaccgccgtgctgacaaagcctagcgcctcttttgagcaggccagcagcggcagccagatcccttttgatccttacgccaccgacagcgacgagcacctggaagtgctgctgaatagccagagccctctgggcaagatgagcgacgtgacactgctgaagatcggcagcgaggaaccccacttcgatggcatcatggatggcttttctggaaaggccgccgaggacctgttcaacgcccacgaaattctgcctggccctctgagccctatgcagacccagtttagccctagcagcgtggactctaatggcctgcagctgtcctttaccgagagcccctgggagacaatggaatggctggacctgaccccccctaatagcacacctggattttctgccctgaccaccagcagcccctctatcttcaatatcgacttcctggacgtgaccgacctgaacctgaacagcagcatggacctgcatctgcagcagtggtga

SEQ ID NO: 5 - protein, 220 aa, synthetic construct
MSLVGGFPHHPVVHHEGYPFAAAAAAAAAAAASRCSHEENPYFHGWLIGHPEMSPPDYSMALSYSPEYASGAAGLDHSHYGGVPPGAGPPGLGGPRPVKRRGTANRKERRRTQSINSAFAELRECIPNVPADTKLSKIKTLRLATSYIAYLMDLLAKDDQNGEAEAFKAEIKKTDVKEEKRKKELNEILKSTVSSNDKKTKGRTGWPQHVWALELKQGSG

SEQ ID NO: 6 - protein, 986 aa, synthetic construct
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 7 - protein, 18 aa, synthetic construct
EGRGSLLTCGDVEENPGP

SEQ ID NO: 8 - protein, 19 aa, synthetic construct
ATNFSLLKQAGDVEENPGP

SEQ ID NO: 9 - protein, 20 aa, synthetic construct
QCTNYALLKLAGDVESNPGP

SEQ ID NO: 10 - protein, 22 aa, synthetic construct
VKQTLNFDLLKLAGDVESNPGP

SEQ ID NO: 11 - DNA, 63 bp, synthetic construct
  source 1..63: mol_type = other DNA
gccacaaacttcagcctgctgaaacaggctggcgacgtggaagagaaccctggcccttctaga

SEQ ID NO: 12 - DNA, 1413 bp, synthetic construct
  source 1..1413: mol_type = other DNA
ttcctgttgcagataagcccagcttagcccagctgaccccagaccctctcccctcactccccccatgtcgcaggatcgagaccctgaggcagacagcccgttcaccaagccccccgccccgcccccatcaccccgtaaacttctcccagcctccgccctgccctcacccagcccgctgttccccaagcctcgctccaagcccacgccacccctgcagcagggcagccccagaggccagcacctatccccgaggctggggtcgaggctcggccccgcccctgcctctgcaacttgagcctggctgcgacccctgctctgacgtctcggaaaattcccccttgcccaggcccttgggggagggggtgcatggtatgaaatggggctgagacccccggctgggggcagaggaacccgccagagaaggagccaaattaggcttctgtttccctgatctggcactccaaggggacacgccgacagcgacagcagagacatgctggaaaggtacaagctcatccctggcaagcttcccacagctggactggggctccgcgttactgcacccagaagttccatggggggcggagcccgactctcaggctcttccgtggtccggggactggacagacatggcgtgcacagcctgggactcttggagcggcgcctcgcagaccctgggccccgcccctctcggcccgggccccatccccgccgccggctccgaaggcgccgcgggccagaactgcgtccccgtggcgggagaggccacctcgtggtcgcgcgcccaggccgccgggagcaacaccagctgggactgttctgtggggcccgacggcgatacctactggggcagtggcctgggcggggagccgcgcacggactgtaccatttcgtggggcgggcccgcgggcccggactgtaccacctcctggaacccggggctgcatgcgggtggcaccacctctttgaagcggtaccagagctcagctctcaccgtttgctccgaaccgagcccgcagtcggaccgtgccagtttggctcgatgccccaaaactaaccaccgaggtcccattcagctgtggcagttcctcctggagctgctccacgacggggcgcgtagcagctgcatccgttggactggcaacagccgcgagttccagctgtgcgaccccaaagaggtggctcggctgtggggcgagcgcaagagaaagccgggcatgaattacgagaagctgagccggggccttcgctactactatcgccgcgacatcgtgcgcaagagcggggggcgaaagtacacgtaccgcttcgggggccgcgtgcccagcctagcctatccggactgtgcgggaggcggacggggagcagagacacaataaaaattcccggtcaaacctcaaaaaaaaaaaaaaa

SEQ ID NO: 13 - DNA, 640 bp, synthetic construct
  source 1..640: mol_type = other DNA
tcgggcctccgaaaccatgaactttctgctgtcttgggtgcattggagccttgccttgctgctctacctccaccatgccaagtggtcccaggctgcacccatggcagaaggaggggggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatct

SEQ ID NO: 14 - protein, 248 aa, synthetic construct
ACTAWDSWSGASQTLGPAPLGPGPIPAAGSEGAAGQNCVPVAGEATSWSRAQAAGSNTSWDCSVGPDGDTYWGSGLGGEPRTDCTISWGGPAGPDCTTSWNPGLHAGGTTSLKRYQSSALTVCSEPSPQSDRASLARCPKTNHRGPIQLWQFLLELLHDGARSSCIRWTGNSREFQLCDPKEVARLWGERKRKPGMNYEKLSRGLRYYYRRDIVRKSGGRKYTYRFGGRVPSLAYPDCAGGGRGAETQ

SEQ ID NO: 15 - protein, 191 aa, synthetic construct
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 16 - DNA, 20 bp, synthetic construct
  source 1..20: mol_type = other DNA
aggctcacttcgagaacagg

SEQ ID NO: 17 - DNA, 19 bp, synthetic construct
  source 1..19: mol_type = other DNA
attgcgaatacgctgctgt

SEQ ID NO: 18 - DNA, 22 bp, synthetic construct
  source 1..22: mol_type = other DNA
acatcatgttttaccgcctgag

SEQ ID NO: 19 - DNA, 23 bp, synthetic construct
  source 1..23: mol_type = other DNA
tttgtggttattgaactctggct

SEQ ID NO: 20 - DNA, 22 bp, synthetic construct
  source 1..22: mol_type = other DNA
gtgacgatcacagaagccaagg

SEQ ID NO: 21 - DNA, 20 bp, synthetic construct
  source 1..20: mol_type = other DNA
tgacagcaggcagccaaacg

SEQ ID NO: 22 - DNA, 21 bp, synthetic construct
  source 1..21: mol_type = other DNA
gattattgctccccctgagcg

SEQ ID NO: 23 - DNA, 22 bp, synthetic construct
  source 1..22: mol_type = other DNA
gtgtaaggtagccgcctcagaa

SEQ ID NO: 24 - DNA, 20 bp, synthetic construct
  source 1..20: mol_type = other DNA
gcgaaggcaacagtcgattc

SEQ ID NO: 25 - DNA, 20 bp, synthetic construct
  source 1..20: mol_type = other DNA
cccaagttccggtgtgactc

SEQ ID NO: 26 - DNA, 23 bp, synthetic construct
  source 1..23: mol_type = other DNA
ggctgaagactgccttgaatgac

SEQ ID NO: 27 - DNA, 20 bp, synthetic construct
  source 1..20: mol_type = other DNA
cgtggcagcaccttcaaaga

SEQ ID NO: 28 - DNA, 22 bp, synthetic construct
  source 1..22: mol_type = other DNA
ggcacagtcaaggctgagaatg

SEQ ID NO: 29 - DNA, 21 bp, synthetic construct
  source 1..21: mol_type = other DNA
atggtggtgaagacgccagta

SEQ ID NO: 30 - DNA, 20 bp, synthetic construct
  source 1..20: mol_type = other DNA
ggaaaacaatgcccagactc

SEQ ID NO: 31 - DNA, 20 bp, synthetic construct
  source 1..20: mol_type = other DNA
gtggaatacgtccaggtggc

SEQ ID NO: 32 - DNA, 20 bp, synthetic construct
  source 1..20: mol_type = other DNA
aagatggtgcgacaaacaag

SEQ ID NO: 33 - DNA, 20 bp, synthetic construct
  source 1..20: mol_type = other DNA
agagagcatcgaaggtggag

SEQ ID NO: 34 - DNA, 19 bp, synthetic construct
  source 1..19: mol_type = other DNA
agacgcctccaggatctgt

SEQ ID NO: 35 - DNA, 21 bp, synthetic construct
  source 1..21: mol_type = other DNA
tcttcaacagctgcttcttcc

SEQ ID NO: 36 - DNA, 21 bp, synthetic construct
  source 1..21: mol_type = other DNA
gctggtcaccaacaatcccta

SEQ ID NO: 37 - DNA, 21 bp, synthetic construct
  source 1..21: mol_type = other DNA
cgtcaaaggcactatcggtgg

SEQ ID NO: 38 - DNA, 20 bp, synthetic construct
  source 1..20: mol_type = other DNA
gccaaagactgtgggtctca

SEQ ID NO: 39 - DNA, 20 bp, synthetic construct
  source 1..20: mol_type = other DNA
gaaggtcgctggtccacaat

SEQ ID NO: 40 - DNA, 21 bp, synthetic construct
  source 1..21: mol_type = other DNA
catgttcagctttgtggacct

SEQ ID NO: 41 - DNA, 20 bp, synthetic construct
  source 1..20: mol_type = other DNA
gcagctgacttcagggatgt

SEQ ID NO: 42 - DNA, 22 bp, synthetic construct
  source 1..22: mol_type = other DNA
ctcatagtcgtatcaggggtcg

SEQ ID NO: 43 - DNA, 21 bp, synthetic construct
  source 1..21: mol_type = other DNA
acacagtcgttttctgtccac

SEQ ID NO: 44 - DNA, 19 bp, synthetic construct
  source 1..19: mol_type = other DNA
tcaccaccatggagaaggc

SEQ ID NO: 45 - DNA, 20 bp, synthetic construct
  source 1..20: mol_type = other DNA
gctaagcagttggtggtgca

SEQ ID NO: 46 - DNA, 21 bp, synthetic construct
  source 1..21: mol_type = other DNA
gaaaacaatgcccagactcaa

SEQ ID NO: 47 - DNA, 18 bp, synthetic construct
  source 1..18: mol_type = other DNA
tgcgcgtggtctgtgtta

SEQ ID NO: 48 - DNA, 19 bp, synthetic construct
  source 1..19: mol_type = other DNA
tgtatccgcatgcaggact

SEQ ID NO: 49 - DNA, 23 bp, synthetic construct
  source 1..23: mol_type = other DNA
ctgtgttatagggactggtggac

(19) United States
(12) Patent Application Publication
(10) Pub. No.: US 2024/0342312 A1
(43) Pub. Date: Oct. 17, 2024

(54) A SHORTENED P63-PROTEIN DOMAIN TO ENHANCE HUMAN CARDIAC REPROGRAMMING

(71) Applicant: Baylor College of Medicine, Houston, TX (US)

(72) Inventors: Todd Rosengart, Houston, TX (US); Jayapratap Pinnamaneni, Houston, TX (US); Vivek P. Singh, Houston, TX (US); Jiangchang Yang, Pearland, TX (US)

(73) Assignee: Baylor College of Medicine, Houston, TX (US)

(21) Appl. No.: 18/580,421

(22) PCT Filed: Aug. 3, 2022
(86) PCT No.: PCT/US2022/074492

§ 371 Date: Jan. 18, 2024

Related U.S. Application Data
(60) Provisional application No. 63/228,671, filed on Aug. 3, 2021.

(51) Int. Cl. A61K 48/00; A61K 38/17; A61K 38/18; A61K 45/06; A61P 9/04; C07K 14/82; C07K 16/18; C12N 15/113; C12N 15/86

(52) U.S. Cl. CPC A61K 48/0058; A61K 38/1709; A61K 38/1866; A61K 45/06; A61K 48/0033; A61K 48/0075; A61P 9/04; C07K 14/82; C07K 16/18; C12N 15/113; C12N 15/86; C12N 2310/14; C12N 2310/531; C12N 2740/15043

(57) ABSTRACT

Embodiments of the disclosure include methods and compositions for in situ cardiac cell regeneration, including transdifferentiation of cardiac cells to cardiomyocytes. In particular embodiments, in situ cardiac cell regeneration encompasses delivery of p63-TID and one or both of Hand2 and myocardin, and in specific embodiments further includes one or more of Gata4, Mef2c, and Tbx5, and/or one or more of ETV2 and VEGF. In specific aspects of the disclosure, adult cardiac fibroblasts are reprogrammed into cardiomyocytes using viral vectors that harbor p63-TID and one or both of the transcription factors Hand2 and myocardin.

### CROSS-REFERENCE TO RELATED APPLICATIONS

This application claims priority to U.S. Provisional Patent Application Ser. No. 63/228,671, filed Aug. 3, 2021, which is incorporated by reference herein in its entirety.

### STATEMENT REGARDING FEDERALLY SPONSORED RESEARCH OR DEVELOPMENT

This invention was made with government support under HL152280 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Aug. 1, 2022, is named BAYM_P0344WO_SequenceListing and is 51,424 bites in size.

### TECHNICAL FIELD

Embodiments of the disclosure include at least the fields of molecular biology, cell biology, cell therapy, and medicine, including cardiac medicine.

### BACKGROUND

Despite medical and surgical innovations, heart disease remains the number one cause of death in the world. Given the poor regenerative capacity of the heart following myocardial infarction and the irreversible loss of cardiomyocytes, the replacement of cardiomyocytes by forming induced pluripotent stem cells or stimulating direct cellular reprogramming are potential therapeutic strategies that hold great promise. Both technologies are rooted in the idea that endogenous fibroblasts within the infarcted myocardium can be reprogrammed into functional cardiomyocytes. Several institutions have reported that a cocktail of transcription factors, most notably Gata4, Mef2c, and Tbx5, can be used to reprogram fibroblasts into cardiomyocytes in vitro. Nevertheless, the major obstacle in the implementation of this therapy is the low efficiency of reprogramming.

The present disclosure provides solutions to a long-felt need in the art for efficient and effective repair of cardiac tissue.

### BRIEF SUMMARY

Embodiments of the disclosure include methods and compositions for the treatment of any medical condition related to the mammalian heart. In specific embodiments, the disclosure concerns treatment of one or more cardiac medical conditions with therapeutic compositions that affect endogenous cells or tissue in the heart. In particular embodiments, therapy is provided to an individual in need thereof, such as when the individual has a need for in situ or in vivo therapy of endogenous cardiac tissue because of a cardiac medical condition or risk thereof. In specific embodiments, the individual has cardiac cellular or cardiac tissue damage from a cardiac medical condition.

In particular embodiments, delivery of certain composition(s) to cells in situ or in vivo in the individual allows regeneration of cardiomyocytes by allowing reprogramming of endogenous non-cardiomyocyte cells to become cardiomyocytes. Upon delivery of a therapeutically effective amount of one or more composition(s) to the individual, the composition(s) provide improvement of the condition at least in part, such as by allowing regeneration of cardiac tissue or cells therein. In specific embodiments, the composition(s) comprise one or more p63-Transactivation Inhibitory domain (p63-TID) polypeptides (or a functional fragment and/or a functional derivative thereof) and/or nucleic acid encoding the same, and/or other factors the downregulation of which would enhance the reprogrammability or plasticity of target cells. In certain embodiments, an individual may also be provided with one or more cardiac cell reprogramming factors (which may or may not be transcription factors) and these may or may not be provided at the same time or in the same composition as p63-TID. In particular embodiments, the composition(s) comprise Hand2, myocardin, or both. In certain embodiments, an individual is also provided with one or more chromatin destabilizing agents. In particular embodiments, the composition(s) comprise p63-TID, Hand2, myocardin, VEGF, and/or ETV2. In particular embodiments, VEGF and/or ETV2 are provided to cells before p63-TID, Hand2, and/or myocardin. In particular embodiments, VEGF and/or ETV2 are provided at the same time as p63-TID, Hand2, and/or myocardin. In certain embodiments, VEGF and/or ETV2 function synergistically with p63-TID, Hand2, and/or myocardin for cardiac cell reprogramming.

In particular embodiments of the disclosure, p63-TID (or a functional fragment and/or a functional derivative thereof) increases the transdifferentiation efficiency of cardiac cells (such as fibroblasts) into cardiomyocytes. As described herein, provision of p63-TID (or a functional fragment and/or a functional derivative thereof) is a novel therapeutic intervention that allows the reprogramming of fibroblasts (for example) into cardiomyocytes at higher efficiencies. Provision of p63-TID (or a functional fragment and/or a functional derivative thereof) is a unique intervention that is a clinically relevant therapy for the treatment of any cardiac medical condition, including heart failure.

In specific embodiments, any p63-TID (or a functional fragment and/or a functional derivative thereof) and/or one or more cardiac cell reprogramming factors and/or one or more chromatin destabilizing agents and/or one or more anti-fibrotic agents and/or one or more angiogenic factors act synergistically with each other and are provided to an individual in need thereof, regardless of whether or not they are in the same composition as p63-TID or are provided at the same time as p63-TID.

In specific embodiments, an individual in need thereof receives one or more anti-fibrotic agents, such as one or more anti-Snail agents (for example, siRNA, antibody, small molecule such as ITD-1, etc.).

In some embodiments, there is a method of in vivo reprogramming of cardiac cells, comprising the step of providing a therapeutically effective amount of one or more compositions to the heart of an individual, wherein said one or more compositions comprises p63-TID (or a functional fragment and/or a functional derivative thereof).

In embodiments of the disclosure, methods comprise the step of providing to the individual an effective amount of one or more cardiac cell reprogramming factors, which may be a polypeptide, peptide, nucleic acid or mixture thereof. In specific embodiments, the one or more cardiac cell reprogramming factors is Hand2, myocardin, Gata4, Mef2c, Tbx5, Mesoderm posterior protein 1 (Mesp1), miR-133, miR-1, Oct4, Klf4, c-myc, Sox2, Brachyury, Nkx2.5, ets variant 2 (ETS2; also referred to as ETV2), VEGF, ESRRG, Mrtf-A, MyoD, ZFPM2, miR-590, miR-208, miR-499, or a combination thereof. In certain embodiments, the one or more cardiac cell reprogramming factors is one or both of Hand2 and myocardin nucleic acids or polypeptides. In some cases, the one or both of Hand2 and myocardin nucleic acids or polypeptides are in the same or different composition as the p63-TID (or a functional fragment and/or a functional derivative thereof). In specific embodiments, the one or more compositions comprise the nucleic acids of p63-TID (or a functional fragment and/or a functional derivative thereof) and Hand2, the nucleic acids of p63-TID (or a functional fragment and/or a functional derivative thereof) and myocardin, and/or the nucleic acids of p63-TID (or a functional fragment and/or a functional derivative thereof), Hand2, and myocardin.

In certain embodiments, p63-TID (or a functional fragment and/or functional derivative thereof) is provided before the one or more cardiac cell reprogramming factors. In particular embodiments of the method, an effective amount of one or more chromatin destabilizing agents are provided to the individual. In particular embodiments, the one or more chromatin destabilizing agents are provided to the individual prior to when the p63-TID (or a functional fragment and/or a functional derivative thereof) is provided to the individual. In some embodiments, the one or more chromatin destabilizing agents are provided to the individual prior to when the p63-TID (or a functional fragment and/or a functional derivative thereof) is provided to the individual, and wherein the p63-TID (or a functional fragment and/or a functional derivative thereof) is provided to the individual prior to when the one or more cardiac cell reprogramming factors are provided to the individual.

In certain embodiments, the cardiac cells are fibroblasts, endothelial cells, myoblasts, progenitor cells, stem cells, myofibroblasts, or a combination thereof. The cardiac cell may be a dividing cell or a non-dividing cell.

In particular embodiments, the p63-TID (or a functional fragment and/or a functional derivative thereof) comprises a nucleic acid and said nucleic acid is comprised on one or more vectors. One or more cardiac cell reprogramming factors may comprise a nucleic acid and the nucleic acid may be comprised on one or more vectors. In specific embodiments, one or more chromatin destabilizing agents comprise a nucleic acid and the nucleic acid is comprised on one or more vectors. In some embodiments, the nucleic acids are comprised on separate vectors or on the same vector. In certain cases, the vector is a viral vector or a non-viral vector, such as a nanoparticle, plasmid, liposome, or a combination thereof. In a specific embodiment, the viral vector is an adenoviral, lentiviral, retroviral, adeno-associated viral vector, or episomal (non-integrating) vectors. In particular embodiments, p63-TID (or a functional fragment and/or a functional derivative thereof), Hand2, and/or myocardin nucleic acids are comprised on a lentiviral vector or are comprised on an adenoviral vector or are a modified mRNA molecule. In any vector encompassed by the disclosure, there may be a cell-specific promoter, such as a fibroblast-specific promoter.

In specific embodiments, any method encompassed by the disclosure comprises the step of delivering to the individual an additional cardiac therapy, such as one that comprises drug therapy, surgery, ventricular assist device (VAD) implantation, video assisted thoracotomy (VAT)coronary bypass, percutaneous coronary intervention (PCI), or a combination thereof.

Any of the compositions encompassed by the disclosure may be provided to the individual in a suitable delivery route, including systemic or local delivery. In specific embodiments, the delivery is local to the heart, and in specific embodiments, the providing step is further defined as injecting the compound(s) into the heart.

In certain embodiments, there is a composition comprising one or more nucleic acid vectors, the vectors comprising p63-TID (or a functional fragment and/or a functional derivative thereof) and comprising one or more cardiac cell reprogramming factors. In some cases, the vector comprising p63-TID (or a functional fragment and/or a functional derivative thereof) is the same vector that comprises one or more cardiac cell reprogramming factors. In certain embodiments, the vector comprising p63-TID (or a functional fragment and/or a functional derivative thereof) is a different vector than the vector that comprises one or more cardiac cell reprogramming factors. In specific embodiments, a vector further comprise one or more chromatin destabilizing agents. The vector that comprises p63-TID (or a functional fragment and/or a functional derivative thereof) may be the same vector that comprises one or more chromatin destabilizing agents. The vector that comprises p63-TID (or a functional fragment and/or a functional derivative thereof) and that comprises one or more cardiac cell reprogramming factors may be the same vector that comprises one or more chromatin destabilizing agents. The vector that comprises p63-TID (or a functional fragment and/or a functional derivative thereof) and that comprises one or more cardiac cell reprogramming factors may be a different vector that comprises one or more chromatin destabilizing agents. The composition that comprises a vector may comprise p63-TID (or a functional fragment and/or a functional derivative thereof) and one or both of Hand 2 and myocardin nucleic acids. In certain embodiments, the composition that comprises a vector comprises p63-TID (or a functional fragment and/or a functional derivative thereof) and Hand2 nucleic acids. In some embodiments, a composition that comprises a vector comprises p63-TID (or a functional fragment and/or a functional derivative thereof) and myocardin nucleic acids or may comprise p63-TID (or a functional fragment and/or a functional derivative thereof), Hand2, and myocardin nucleic acids. In specific embodiments, any composition of the disclosure may comprise one or more anti-fibrotic agents.

In some embodiments there is a kit comprising a composition encompassed by the disclosure, said composition being housed in a suitable container.

In certain embodiments, provided herein are methods of treating a heart condition, comprising: the step of providing a therapeutically effective amount of one or more compositions to the heart of an individual, wherein said one or more compositions comprises, consist essentially of, or consist of: A) a p63-Transactivation Inhibitory domain (p63-TID) polypeptide and/or a functional derivative and/or a functional fragment thereof, and/or a nucleotide encoding the same; wherein the p63-TID polypeptide comprises, consists essentially of, consists of, or is, a sequence that is at least or exactly at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NO: 1; B) a Hand2 polypeptide and/or a functional derivative and/or a functional fragment thereof, and/or a nucleotide encoding the same; C) a myocardin polypeptide and/or a functional derivative and/or a functional fragment thereof, and/or a nucleotide encoding the same; D) an ETV2 polypeptide and/or a functional derivative and/or a functional fragment thereof, and/or a nucleotide encoding the same; and/or E) a VEGF polypeptide and/or a functional derivative and/or a functional fragment thereof, and/or a nucleotide encoding the same. In some embodiments, the method comprises, consists essentially of, or consists of providing A, B, C, and D. In some embodiments, the method comprises, consists essentially of, or consists of providing A, B, C, and E. In some embodiments, D and/or E are provided on the same day as A, B, and C. In some embodiments, D and/or E are provided simultaneously with A, B, and C. In some embodiments, D and/or E are provided before A, B, and C. In some embodiments, D and/or E are provided at least or exactly 1, 2, 3, 4, 5, 6, 7, 8, 9, 10, 11, 12, 13, 14, 15, 16, 17, 18, 19, 20, 21, 22, 23, 24, 25, 26, 27, or 28 days, or any range derivable therein, before A, B, and C. In some embodiments, A, B, and C are provided before D and/or E. In some embodiments, A, B, and C are provided at least or exactly 1, 2, 3, 4, 5, 6, 7, 8, 9, 10, 11, 12, 13, 14, 15, 16, 17, 18, 19, 20, 21, 22, 23, 24, 25, 26, 27, or 28 days, or any range derivable therein, before D and/or E. In some embodiments, A, B, C, D, and/or E are provided in a nanoparticle, plasmid, liposome, viral vector, or any combination thereof. In some embodiments, A, B, C, D, and/or E are provided in a viral vector, wherein the viral vector is an adenoviral, lentiviral, retroviral, or adeno-associated viral vector. In some embodiments, a viral vector is an adenoviral vector. In certain embodiments, one or more compositions comprising A, B, C, D, and/or E are provided to cells in vitro. In certain embodiments, cells provided with A, B, C, D, and/or E in vitro are provided to an individual with a heart condition. In certain embodiments, cells provided with A, B, C, D, and/or E in vitro are provided directly to the heart of an individual with a heart condition.

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1 A-B shows that the p63 isoform ΔNp63α and histone deacetylase 1 (HDAC1) physically interact with each other, and p63-TID disrupts binding between them. FIG. 1 A, ΔNp63α-FLAG and HDAC1-GFP were over expressed (O.E) in immortalized rat cardiac fibroblasts. Co-IP performed using anti-FLAG antibody followed by immunoblotting with antibodies against HDAC1 and p63. FIG. 1 B, Overexpression of p63-TID interferes p63/HDAC1 interactions. HDAC1-GFP, ΔNP63α FLAG with or without TID vectors were co-expressed in human 293T cells and Co-IP was performed by using indicated antibodies.

FIGS. 2 A-J show an experimental design and the results thereof for differentiation of human cardiac fibroblasts. FIG. 2 A shows a schematic depiction of experimental design to determine the effect of p63-TID on iCM reprogramming in human cardiac fibroblasts (Real-Time Quantitative Reverse Transcription PCR (qRT-PCR); Immunofluorescence (IF); Fluorescence-activated cell sorting (FACS). FIG. 2 B, is FACS data showing percentage of cardiac troponin T-positive (cTnT+) cells 14 days after transduction of indicated lentiviral vectors in human cardiac fibroblasts. (n=3, Data is presented as mean±SEM **p<0.01 Vs shNT *p<0.05 Vs GMT). FIG. 2 C, displays mRNA expression levels of cardiac and fibroblast marker genes in above treated cells as assessed by qRT-PCR (n=3; Error bars refer mean±SEM; *P<0.05 Vs GMT, **P<0.001 vs shNT). FIG. 2 D, displays representative immunofluorescence staining for (DAPI) (blue), (FITC) (green), and (red) cardiomyocyte markers cTnT (bar=100 m, images were captured at 1OX magnification). FIG. 2 E, displays representative immunofluorescence staining for (DAPI) (blue), (FITC) (green), and (red) cardiomyocyte marker and a-actinin (bar=100 m, images were captured at 1OX magnification). FIG. 2 F, displays cardiomyocyte marker gene (cTnT, Gja1, and Myh6) expression assessed by qRT-PCR after indicated treatments (n=3, ****p<0.0001). FIG. 2 G, displays fibroblast marker gene (Collal, and Postn) expression assessed by qRT-PCR after indicated treatments (n=3, ****p<0.0001). FIG. 2 H, displays representative flow cytometry plots for cardiac troponin T positive (cTnT+) human cardiac fibroblasts 2 weeks after their treatment with shp63 or p63-TID with or without Hand2/Myocardin (H/M) (left panel), and quantification (right panel) of the percentage of cTnT+ cells treated as indicated, as assessed by flow cytometry (n=3; ****p<0.0001). FIG. 2 I, displays quantification of cells expressing cardiomyocyte markers cTnT+ and α-actinin+ (e.g., imaged in FIG. 2 E) two weeks after indicated treatments, as assessed by immunofluorescence labelling (n=3, ***p<0.001, **p<0.01). FIG. 2 J, displays representative high-magnification images of cTnT and α-actinin staining in cells treated with p63-TID and H/M demonstrating sarcomeric structures, most clearly visible in α-actinin labeled cells. Scale bar=25 m (left panel), and quantification (right panel) of cells with well-developed sarcomeres as a percentage of total α-actinin+ cells after 4 weeks of shp63+ GMT, shp63+ H/M, or p63-TID+ H/M transduction (n=3; **p<0.01, ***p<0.001).

FIG. 3 shows p63-TID administration in rat cardiac fibroblasts yielded better iCM reprogramming outcomes. shown are mRNA expression levels of indicated cardiac and fibroblast marker gene mRNA expression 2 weeks after reprogramming factors administration, as indicated above, in rat cardiac fibroblasts, assessed by qRT-PCR (n=3, all data are presented as mean±SEM. *P<0.05 Vs GMT, **P<0.001 vs shNT).

FIGS. 4 A-B provides an exemplary p63-TID sequence and an exemplary construct design for a vector comprising the same. FIG. 4 A, depicts one example of a lentiviral vector map comprising p63-TID nucleic acid sequence (TID pp3018). Such an example of a vector may be utilized in reprogramming assays, such as qPCR, FACS, and/or immunofluorescence. FIG. 4 B shows a vector utilized in co-IP studies in which TID nucleotide sequence was cloned in a pcDNA3.1 vector backbone.

FIG. 5, displays the functional efficacy of human cardiac fibroblast reprograming after co-culture with neonatal rat cardiomyocytes. Adult human cardiac fibroblasts were treated with lentivirus expressing GMT (left), shp63 in combination with Hand2/Myocardin (H/M) (middle) or p63-TID+ Hand2/Myocardin (H/M; p63TID+ H/M) (right). One week after initial transduction, these human cardiac fibroblasts were co-cultured with (untreated) neonatal rat cardiomyocytes (negative for GFP [green fluorescent protein]). The top row depicts representative immunofluorescence demonstrating (green) GFP expression by human cardiac fibroblasts treated with GMT (left), shp63+ H/M (middle) or p63-TID+ H/M (right) after 4 weeks in co-culture with (non-transduced) neonatal rat cardiomyocytes. Bar=100 μm. The middle and bottom rows depict representative peaks from GFP+ human cardiac fibroblasts treated with GMT, shp63+ H/M and p63-TID+ H/M after 4 weeks of co-culture, reflecting contraction (top row) and Ca²⁺ transients (bottom row). Contractility parameters were not observed in cells treated with GMT alone. Bar=1 s.

FIGS. 6 A-D, depict dose-based efficacy of p63-TID comparative to shp63 in enhancing human cardiac reprogramming. FIG. 6 A, depicts FLAG co-immunoprecipitation assay in 293T cells transfected with HDAC1, p63-FLAG and/or p63-TID vectors at three different p63-TID dosages showing increasing interference in p63-HDAC1 binding as a function of p63-TID dosage. Beta-actin was used as loading control. IB=immunoblot, IP=immunoprecipitation. FIG. 6 B, depicts dosage screening of p63-TID in human cardiac fibroblasts, qRT-PCR analysis of cardiac Troponin T (cTnT) marker 2 weeks after human cardiac fibroblasts were treated with 20, 50 or 100 MOI of lentiviral vector expressing p63-TID (n=3; ***p<0.001, **p<0.01). Control: lentiviral GFP vector, 20 MOI. FIG. 6 C, depicts cardiomyocyte marker gene expression in human cardiac fibroblasts as assessed by qRT-PCR two weeks after indicated treatments using p63-TID vector at a multiplicity of infection (MOI) of 50 (n=3, H/M=20 MOI; *p<0.05. versus shp63 +H/M). FIG. 6 D, depicts fibroblast marker gene expression in human cardiac fibroblasts as assessed by qRT-PCR two weeks after indicated treatments using p63-TID vector at a multiplicity of infection (MOI) of 50 (n=3, H/M=20 MOI; *p<0.05. versus shp63 +H/M).

FIGS. 7 A-D, shows experimental designs and the results thereof for differentiation of human cardiac fibroblasts. FIG. 7 A, depicts an experimental design for delayed delivery (i.e. sequential and temporally distinct deliveries) of adenoviral vectors encoding GFP (adGFP; control), Ets variant 2 (ETV2), or Vascular endothelial growth factor (VEGF), and adenoviral vectors encoding GFP (GFP; control); GMT (Gata4 (GATA binding protein 4), Mef2c (myocyte enhancer factor 2c), and Tbx5 (t-box transcription factor 5)), GMTd (Gata4, Mef2c, and TEAD1 (TEA domain family member 1), or TIDH/M (p63-TID+ H/M). FIG. 7 B, depicts relative mRNA expression for cTnT following an experimental design as outlined in FIG. 7 A (n=3). FIG. 7 C, depicts an experimental design for simultaneous delivery (i.e. single or multiple compositions delivered during a single setting) of adenoviral vectors encoding GFP (adGFP; control), Ets variant 2 (ETV2), or Vascular endothelial growth factor (VEGF), and adenoviral vectors encoding GFP (GFP; control); GMT (Gata4, Mef2c, and Tbx5), GMTd (Gata4, Mef2c, and TEAD1), or TIDH/M (p63-TID+ H/M). FIG. 7 D, depicts relative mRNA expression for cTnT following an experimental design as outlined in FIG. 7 C (n=3).

### DETAILED DESCRIPTION

Some embodiments of the disclosure may consist of or consist essentially of one or more elements, method steps, and/or methods of the disclosure. It is contemplated that any method or composition described herein can be implemented with respect to any other method or composition described herein.

The term “cardiac medical condition” as used herein refers to any medical condition that affects heart tissue, including that affects heart function.

The term “chromatin destabilizing agent” as used herein refers to one or more compounds that facilitate access of one or more factors to condensed genomic DNA.

The term “cardiac cell reprogramming factor” as used herein refers to one or more compositions that enhance or facilitate the transdifferentiation of a differentiated cell in the heart to a cardiomyocyte.

Embodiments of the disclosure include methods and compositions for the therapy or prevention of any cardiac medical condition in which it would be therapeutic to increase the number of cardiomyocytes in the heart. In specific embodiments, in vivo cells in the heart are reprogrammed to become cardiomyocytes. In particular embodiments, this is achieved at least in part by providing an effective amount of p63-TID (or a functional fragment and/or a functional derivative thereof). In particular embodiments, nucleic acids and/or peptides and/or polypeptides are delivered directly to the heart to allow for reprogramming of non-cardiomyocyte cells in the heart to become cardiomyocytes.

### I. p63-TID Compositions

Embodiments of the disclosure include methods and compositions related to p63-Transactivation Inhibitory domain (p63-TID). In particular embodiments, p63-TID (or a fragment and/or a derivative thereof) acts as a dominant negative inhibitor of p63 activity of any kind. In particular embodiments, p63-TID (or a fragment and/or a derivative thereof) acts as a dominant negative inhibitor of isoforms Tap63 and/or ΔNp63. In particular embodiments, p63-TID acts to inhibit and/or silence the epigenetic effects of p63, and/or enhance cardiogenic reprogramming gene activation. In particular embodiments, p63-TID promotes the downregulation of fibrogenic genes known to impede cardio-differentiation. In particular embodiments, p63-TID provides an HDAC-directed reprogramming strategy that avoids activation and/or silencing of genes unrelated to the desired cardio-differentiation effects, including but not limited to potential oncogenes. A composition comprising p63-TID (or a fragment and/or a derivative thereof) and/or a polynucleotide vector comprising a nucleic acid sequence encoding the same, is utilized for effective treatment of any cardiac medical condition.

An example of a p63-TID polypeptide is as follows: MTTIYQIEHYSMDDLASLKIPEQFRHAIWKGILDHRQLHEFSSPSHLLRTPSSASTVSVGSSET RGERVIDA (SEQ ID NO: 1)

An example of a nucleic acid sequence that may encode p63-TID is as follows:

(SEQ ID NO: 2)
ATGACCACCATCTATCAGATTGAGCATTACTCCATGGATGATCTGGCAAG
TCTGAAAATCCCTGAGCAATTTCGACATGCGATCTGGAAGGGCATCCTGG
ACCACCGGCAGCTCCACGAATTCTCCTCCCCTTCTCATCTCCTGCGGACC
CCAAGCAGTGCCTCTACAGTCAGTGTGGGCTCCAGTGAGACCCGGGGGAG
CGTGTTATTGATGCTTAA

p63-TID may be isolated from human cells, and therefore no longer residing in nature, or it may be recombinant, in certain embodiments. As referred to herein, when the native sequence of SEQ ID NO:1 is generated by recombinant means, the resultant polypeptide may be referred to as a recombinant p63-TID. Another example of a recombinant p63-TID includes a label or tag. Embodiments of p63-TID include functional derivatives and/or functional fragments thereof, and the derivative or fragment may be considered functional if it has the ability to allow reprogramming of cells upon exposure to the fragment, either alone or in combination with one or more cardiac cell reprogramming factors. Such an activity may be measured by any suitable means, including by qPCR, flow cytometry, immunofluorescence, and/or beating assays, for example. In specific embodiments, the p63-TID or functional fragment and/or functional derivative thereof is soluble. The p63-TID or functional fragment and/or functional derivative thereof may or may not be comprised in a fusion protein.

p63-TID proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. A p63-TID coding region may be amplified and/or expressed using the techniques disclosed herein or as would be known to those of ordinary skill in the art. Alternatively, various commercial preparations of proteins, polypeptides and peptides are known to those of skill in the art.

In certain embodiments a p63-TID (or a fragment and/or a derivative thereof) proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide. Biological functional equivalents of p63-TID, including such derivatives and fragments, may be employed. As modifications and/or changes may be made in the structure of p63-TID polynucleotides and and/or proteins according to the present invention, while obtaining molecules having similar or improved characteristics, such biologically functional equivalents are also encompassed within the present invention.

In some embodiments, p63-TID is utilized in protein form, including as SEQ ID NO: 1. In cases wherein a functional derivative or fragment of p63-TID is utilized, a p63-TID functional derivative or fragment thereof may comprise 1, 2, 3, 4, 5, 6, 7, 8, 9, 10, 11, 12, 13, 14, 15, 16, 17, 18, 19, or 20 or more amino acid alterations compared to SEQ ID NO: 1. The p63-TID functional derivative or fragment thereof may comprise an N-terminal truncation of SEQ ID NO: 1, for example wherein the truncation is no more than 1, 2, 3, 4, 5, 6, 7, 8, 9, 10, 12, 15, 20, 25, 30, 35, 40, 45, 50, 55, or 60 amino acids or wherein the truncation is at least 1, 2, 3, 4, 5, 6, 7, 8, 9, 10, 12, 15, 20, 25, 30, 35, 40, 45, 50, 55, or 60 amino acids. The p63-TID functional derivative or fragment thereof may comprise a C-terminal truncation of SEQ ID NO: 1, such as wherein the truncation is no more than 1, 2, 3, 4, 5, 6, 7, 8, 9, 10, 12, 15, 20, 25, 30, 35, 40, 45, 50, 55, or 60 amino acids or is at least 1, 2, 3, 4, 5, 6, 7, 8, 9, 10, 12, 15, 20, 25, 30, 35, 40, 45, 50, 55, or 60 amino acids. The p63-TID functional derivative or fragment thereof may comprise an internal deletion in SEQ ID NO: 1, such as wherein the internal deletion is no more than 1, 2, 3, 4, 5, 6, 7, 8, 9, 10, 12, 15, 20, 25, 30, 35, 40, 45, 50, 55, or 60 amino acids or is at least 1, 2, 3, 4, 5, 6, 7, 8, 9, 10, 12, 15, 20, 25, 30, 35, 40, 45, 50, 55, or 60 amino acids. In specific embodiments, a p63-TID functional derivative or fragment thereof may comprise sequence that is at least, or is exactly, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NO: 1.

In some embodiments, p63-TID is utilized in nucleic acid form, such as SEQ ID NO: 2. In cases wherein a functional derivative or fragment of p63-TID nucleic acid is utilized, a p63-TID functional derivative or fragment thereof may comprise 1, 2, 3, 4, 5, 6, 7, 8, 9, 10, 11, 12, 13, 14, 15, 16, 17, 18, 19, or 20 or more differences compared to SEQ ID NO: 2, and these may or may not be in the wobble position. The p63-TID functional derivative or fragment thereof may comprise a truncation at the 5′ end of SEQ ID NO: 2, for example wherein the truncation is no more than 1, 2, 3, 4, 5, 6, 7, 8, 9, 10, 12, 15, 20, 25, 30, 35, 40, 45, 50, 55, 60, 75, 90, 100, 110, 125, or more nucleotides or wherein the truncation is at least 1, 2, 3, 4, 5, 6, 7, 8, 9, 10, 12, 15, 20, 25, 30, 35, 40, 45, 50, 55, 60, 75, 90, 100, 110, 125, or more nucleotides. The p63-TID functional derivative or fragment thereof may comprise a 3′ truncation of SEQ ID NO: 2, such as wherein the truncation is no more than 1, 2, 3, 4, 5, 6, 7, 8, 9, 10, 12, 15, 20, 25, 30, 35, 40, 45, 50, 55, 60, 75, 90, 100, 110, 125, or more nucleotides or is at least 1, 2, 3, 4, 5, 6, 7, 8, 9, 10, 12, 15, 20, 25, 30, 35, 40, 45, 50, 55, 60, 75, 90, 100, 110, 125, or more nucleotides. The p63-TID functional derivative or fragment thereof may comprise an internal deletion in SEQ ID NO: 2, such as wherein the internal deletion is no more than 1, 2, 3, 4, 5, 6, 7, 8, 9, 10, 12, 15, 20, 25, 30, 35, 40, 45, 50, 55, 60, 75, 90, 100, 110, 125, or more nucleotides or is at least 1, 2, 3, 4, 5, 6, 7, 8, 9, 10, 12, 15, 20, 25, 30, 35, 40, 45, 50, 55, 60, 75, 90, 100, 110, 125, or more nucleotides. In specific embodiments, a p63-TID functional derivative or fragment thereof may comprise sequence that is at least, or is exactly, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NO: 2. Any functional fragment and/or functional derivative will retain the biological activity of the full length SEQ ID NO: 1 polypeptide.

In particular embodiments, the disclosure encompasses utilizing p63-TID as a nucleic acid comprised in a recombinant vector(s) comprising nucleic acid sequences that encode p63-TID as a polypeptide. In specific embodiments, the recombinant vector comprises nucleic acid sequence that encodes SEQ ID NO: 1, including all contiguous amino acids therein. In particular aspects, the recombinant vectors are viral vectors (such as lentiviral, adenoviral, adeno-associated viral, or retroviral) or non-viral vectors (such as plasmids, transposons, etc.).

A biological functional equivalent of p63-TID may be produced from a polynucleotide that has been engineered to contain distinct sequences while at the same time retaining the capacity to encode the “wild-type” or standard protein. This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein.

In another example, an p63-TID polynucleotide made be (and encode) a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) may be substituted.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (0.4); threonine (0.7); serine (0.8); tryptophan (0.9); tyrosine (1.3); proline (1.6); histidine (3.2); glutamate (3.5); glutamine (3.5); aspartate (3.5); asparagine (3.5); lysine (3.9); and/or arginine (4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly considered.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (0.4); proline (−0.5±1); alanine (0.5); histidine (0.5); cysteine (1.0); methionine (1.3); valine (1.5); leucine (1.8); isoleucine (1.8); tyrosine (2.3); phenylalanine (2.5); tryptophan (3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

The present disclosure, in many aspects, relies on the synthesis of peptides and polypeptides in cyto, via transcription and translation of appropriate polynucleotides. These peptides and polypeptides will include the twenty “natural” amino acids, and post-translational modifications thereof. However, in vitro peptide synthesis permits the use of modified and/or unusual amino acids. Exemplary, but not limiting, modified and/or unusual amino acids are known in the art.

In addition to the biological functional equivalents discussed above, the present inventors also contemplate that structurally or functionally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule. Such peptidomimetics include compounds that do not incorporate any natural amino acids or amino acid side chains, but are designed based on the p63-TID peptide sequence and have the ability to functionally replace p63-TID.

### II. Pharmaceutical Preparations

Pharmaceutical compositions of the present disclosure comprise an effective amount of p63-TID (or a functional fragment and/or a functional derivative thereof) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one p63-TID (or a functional fragment and/or a functional derivative thereof) will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The p63-TID (or a functional fragment and/or a functional derivative thereof) may comprise different types of carriers depending on whether it is to be administered in solid, liquid or aerosol form, and whether it need to be sterile for such routes of administration as injection. The present invention can be administered intramyocardial, endocardial, epicardial and/or intra-coronary, by direct, catheter, or intracoronary injection, intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The p63-TID (or a functional fragment and/or a functional derivative thereof) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include p63-TID (or a functional fragment and/or a functional derivative thereof), one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the p63-TID (or a functional fragment and/or a functional derivative thereof) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### A. Alimentary Compositions and Formulations

In embodiments of the present disclosure, the p63-TID (or a functional fragment and/or a functional derivative thereof) are formulated to be administered via an alimentary route. Alimentary routes include all possible routes of administration in which the composition is in direct contact with the alimentary tract. Specifically, the pharmaceutical compositions disclosed herein may be administered directly to the heart, although in alternative embodiments the composition is delivered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically- effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### B. Parenteral Compositions and Formulations

In further embodiments, p63-TID (or a functional fragment and/or a functional derivative thereof) may be administered via a parenteral route. As used herein, the term “parenteral” includes routes that bypass the alimentary tract. Specifically, the pharmaceutical compositions disclosed herein may be administered for example, but not limited to intramyocardial, endocardial, epicardial and/or intra-coronary, by direct catheter or intracoronary injection, intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other particular embodiments of the disclosure, the active compound p63-TID (or a functional fragment and/or a functional derivative thereof) may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### III. Embodiments of Methods of Treatment

Embodiments of the present disclosure are directed to methods and/or compositions related to therapy and/or prevention of one or more cardiac-related medical conditions. Embodiments of the present disclosure concern regeneration of tissue, including muscle tissue, such as myocardial tissue, through the reprogramming of existing cells in the heart that are not cardiomyocytes. Certain embodiments relate to reversal of a cardiac medical condition (or improvement of at least one symptom thereof), including at least cardiac disease, cardiomyopathy, cardiotoxicity, congestive heart failure, ischemic heart disease, myocardial infarction, coronary artery disease, cor pulmonale, inflammatory heart disease; inflammatory cardiomegaly; myocarditis; congenital heart disease; rheumatic heart disease, cardiac systolic dysfunction, cardiac diastolic dysfunction, angina, dilated cardiomyopathy, idiopathic cardiomyopathy, or other conditions resulting in cardiac fibrosis, for example.

In particular aspects of the disclosure, cardiomyopathy is the cardiac medical condition to be treated. The cardiac medical condition (including, for example, cardiomyopathy) may be caused by one or more of a variety of characteristics, including, for example, long-term high blood pressure; heart valve problems; heart tissue damage (such as from one or more previous heart attack(s) or chronic or acute and/or recurrent episodes or sequelae of ischemic heart disease); chronic rapid heart rate; metabolic disorders, such as thyroid disease or diabetes; nutritional deficiencies of essential vitamins or minerals, such as thiamin (vitamin B-1), selenium, calcium and/or magnesium; pregnancy; alcohol abuse; drug abuse, including of narcotics or prescription drugs, such as cocaine or antidepressant medications, such as tricyclic antidepressants; use of some chemotherapy drugs to treat cancer (including Adriamycin); certain viral infections; hemochromatosis and/or an unknown cause or undetected cause, i.e. idiopathic cardiomyopathy.

In some cases, methods and compositions of the present disclosure are employed for treatment or prevention of one or more cardiac medical conditions or delay of onset of one or more cardiac medical conditions or reduction of extent of one or more symptoms of one or more cardiac medical conditions. In particular cases, such prevention, delay or onset, or reduction of extent of one or more symptoms, occurs in an individual that is at risk for a cardiac medical condition. Exemplary risk factors include one or more of the following: age, gender (male, although it occurs in females), high blood pressure, high serum cholesterol levels, tobacco smoking, excessive alcohol consumption, sugar consumption, family or personal history, obesity, lack of physical activity, psychosocial factors, diabetes mellitus, overweight, genetic predisposition, and/or exposure to air pollution.

Particular aspects of the disclosure concern delivery of at least one polynucleotide or polypeptide to cardiac tissue for trans-differentiation of certain cells in the tissue. In specific embodiments, a nucleic acid is the active agent, whereas in some embodiments a polypeptide produced from the nucleic acid is the active agent. The tissue may be of any kind, but in specific cases it is cardiac muscle and/or scar tissue. In particular embodiments, methods and compositions of the disclosure allow for differentiation of adult resident cardiac progenitor cells and/or transdifferentiation of non-cardiomyocyte differentiated cells, such as fibroblast cells, into cardiac muscle cells.

Embodiments of the disclosure include delivery of one or more polynucleotides (which may also be referred herein to as nucleic acids) or polypeptides produced therefrom that stimulate transdifferentiation or direct reprogramming of cells (such as muscle cells, including cardiomyocytes) and/or tissue (including cardiac tissue). Particular aspects for such embodiments result in reversal of one or more cardiac medical conditions. Certain aspects for such embodiments result in improvement of at least one symptom of a cardiac medical condition. In exemplary embodiments, the cardiac medical condition is heart failure. The heart failure may be the result of one or more causes, including coronary artery disease and heart attack, high blood pressure, faulty heart valves, cardiomyopathy(such as caused by disease, infection, alcohol abuse and the toxic effect of drugs, such as cocaine or some drugs used for chemotherapy), idiopathic cardiomyopathy and/or genetic factors.

In certain embodiments, one or more polynucleotides are comprised in a viral vector. In certain embodiments, a viral vector is provided at a multiplicity of infection of at least, or exactly 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150, or any range derivable therein. In certain embodiments, a viral vector is provided at a multiplicity of infection of 20, 50, or 100. In certain embodiments, a viral vector is provided at a multiplicity of infection of 50.

Particular but exemplary indications of embodiments of the disclosure include at least applications for 1) heart failure, including congestive heart failure; 2) prevention of ventricular remodeling; and/or 3) cardiomyopathy. Other indications may also include coronary artery disease, ischemic heart disease, valvular heart disease, etc. In specific embodiments, methods and compositions of the disclosure provide cardiomyocyte regeneration that is sufficient to reverse established cardiomyopathy, congestive heart failure, and prevention of ventricular remodeling.

In cases where the individual has cardiomyopathy, the cardiomyopathy may be ischemic or non-ischemic cardiomyopathy. The cardiomyopathy may be caused by long-term high blood pressure, heart valve problems, heart tissue damage from a previous heart attack, chronic rapid heart rate, metabolic disorders, nutritional deficiencies, pregnancy, alcohol abuse, drug abuse, chemotherapy drugs, viral infection, hemochromatosis, genetic condition, elevated cholesterol levels, or a combination thereof. Cardiomyopathy may also have no identified cause, i.e. idiopathic cardiomyopathy.

In certain embodiments, there is a method of regenerating cells at a desired location in an individual, comprising the steps of delivering to the location an effective amount of at least one molecule of p63-TID (or a functional fragment and/or a functional derivative thereof). In some cases, the methods include a mechanistic interaction of p63-TID with an HDAC, for example. In specific embodiments, the molecules are delivered in nucleic acid form, although in specific embodiments one or more of the compositions of the disclosure of p63-TID (or a functional fragment and/or a functional derivative thereof) are polypeptides. In particular embodiments, the delivery location of the composition(s) is at a region of the heart. A delivering step may comprise injection directly into the heart, including directly into the area of damaged tissue; intravenous perfusion; intra-coronary artery myocardium perfusion; intra-artery organ perfusion by catheter; or coronary sinus perfusion catheter, for example.

Embodiments of the disclosure include methods and/or compositions for regeneration of cardiac muscle and reversal of myocardial ischemic injury, for example. In particular embodiments, there are methods for reprogramming of cardiac scar cells (fibroblasts) into adult cardiac muscle cells in mammalian hearts in an individual that has had a cardiac medical condition, such as acute or chronic ischemic injury, for example. In certain embodiments, such methods are achieved with compositions comprising at least p63-TID (or a functional fragment and/or a functional derivative thereof) and, in some cases, one or more cardiac cell reprogramming factors, such as Hand2 and/or myocardin, for example.

Although in particular embodiments an individual is treated in an in vivo or in situ manner, in alternative embodiments the individual is treated with compositions encompassed by the disclosure in an ex vivo manner. In such embodiments, cells that are to be subjected to nucleic acid composition(s) of the disclosure are either obtained from the individual or are obtained from another individual. Such cells are subjected in vitro to the nucleic acid compositions such that they are taken up by the cells, and the cells are then delivered to the individual to be treated.

In particular aspects, an individual is provided with an additional cardiac medical condition therapy.

### IV. Embodiments of Cardiac Cell Reprogramming Factors and Chromatin Destabilizing Agents

Certain embodiments of the present disclosure concern nucleic acids, and some embodiments concern polypeptides or peptides. In certain aspects, nucleic acids include a p63-TID (or a functional fragment and/or a functional derivative thereof). In particular embodiments, p63-TID (or a functional fragment and/or a functional derivative thereof) in either form may or may not be utilized with one or more cardiac cell reprogramming factors and may or may not be used with one or more chromatin destabilizing agents.

### A. Cardiac Cell Reprogramming Factors

In specific embodiments, one or more cardiac cell reprogramming factors are employed in methods of the disclosure, and the factors may or may not be provided at the same time as the p63-TID (or a functional fragment and/or a functional derivative thereof). In specific embodiments, the factors are provided after the individual has received the p63-TID (or a functional fragment and/or a functional derivative thereof), although in some cases they are provided before or at the same time as the agent(s).

The cardiac cell reprogramming factor may or may not be a transcription factor. Although one could use standard methods to test whether or not a certain compound would be effective as a cardiac cell reprogramming factor, in specific embodiments the factor is Hand2, myocardin, Gata4, Mef2c, Tbx5, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, or a combination thereof. One could test whether or not a compound acted as a cardiac cell reprogramming factor by administering it to fibroblasts (e.g., using lentivirus) and perform FACS for cTnT as illustrated elsewhere herein. The factors may be employed as nucleic acids, polypeptides, peptides of specific domains of the factor, or a combination thereof. In specific embodiments, Hand2 and/or myocardin are employed, including as nucleic acids. In particular aspects at least for Hand2 and/or myocardin, the nucleic acid encodes for or comprises a transcribed nucleic acid. In other aspects, a Hand2 and/or myocardin nucleic acid comprises a nucleic acid segment of Hand2 and/or myocardin, respectively, or a biologically functional equivalent thereof. In particular aspects, a Hand2 and/or myocardin nucleic acid encodes a protein, polypeptide, or peptide. An exemplary human Hand2 nucleic acid is at the GENBANK® database of National Center for Biotechnology Information, Accession No. NM_021973, which is incorporated by reference herein in its entirety. An exemplary human myocardin nucleic acid is at GENBANK® Accession Number AY764180, which is incorporated by reference herein in its entirety.

In specific embodiments, a functional fragment of the cardiac cell reprogramming factor nucleic acid or polypeptide is utilized instead of the entire factor nucleic acid or polypeptide. A functional fragment of either Hand2 or myocardin (as an example) is one that is sufficient to allow reprogramming of cells upon exposure to the fragment, either alone with p63-TID (or a functional fragment and/or a functional derivative thereof) or in conjunction with Hand2 or myocardin and p63-TID (or a functional fragment and/or a functional derivative thereof). In specific embodiments, the functional fragment of Hand2 nucleic acid encodes at least 200, 180, 175, 160, 150, 140, 125, 110, 100, 90, 80, 75, 70, 60, 55, 50, 40, 30, 25, or 19 amino acids of the Hand2 polypeptide. In specific embodiments, the functional fragment of myocardin nucleic acid encodes at least 900, 800, 700, 600, 500, 400, 300, 200, 100, or 50 amino acids of the myocardin polypeptide. In some embodiments, a Hand2 polypeptide and a myocardin polypeptide are encoded on the same nucleic acid construct and/or vector. In some embodiments, a Hand2 polypeptide and a myocardin polypeptide are separated by a 2A element. In certain embodiments, a Hand2 polypeptide and a myocardin polypeptide are separated by a P2A element. In certain embodiments, an exemplary Hand2 polynucleotide sequence is and/or is comprised in SEQ ID NO: 3. In certain embodiments, an exemplary myocardin polynucleotide sequence is and/or is comprised in SEQ ID NO: 4.

(SEQ ID NO: 3)
ATGTCTCTCGTGGGCGGATTTCCTCACCACCCTGTGGTGCACCATGAGGGCTATCCTTTTGCTG
CCGCTGCCGCAGCCGCCGCTGCTGCTGCAGCTAGTAGATGTAGCCACGAGGAAAACCCCTACTT
CCACGGCTGGCTGATCGGCCACCCTGAGATGAGCCCTCCAGATTACAGCATGGCCCTGAGCTAC
AGCCCTGAGTATGCTTCTGGAGCCGCTGGACTGGATCACTCTCATTATGGCGGAGTGCCTCCAG
GCGCTGGACCTCCTGGACTGGGAGGACCTAGACCTGTGAAGAGAAGAGGCACCGCCAACCGGAA
AGAGCGGAGAAGAACCCAGAGCATCAATAGCGCCTTCGCCGAGCTGAGAGAATGCATCCCTAAT
GTGCCCGCCGACACCAAGCTGAGCAAGATCAAAACCCTGCGGCTGGCCACCAGCTATATCGCCT
ATCTGATGGACCTGCTGGCCAAGGACGATCAGAATGGCGAGGCCGAGGCCTTCAAGGCCGAGAT
CAAGAAAACCGACGTGAAAGAGGAAAAGCGCAAGAAAGAGCTGAACGAGATCCTGAAGTCCACC
GTGTCCAGCAACGACAAAAAGACCAAGGGCAGAACCGGCTGGCCTCAGCATGTGTGGGCTCTGG
AACTGAAACAGGGCAGCGGC
(SEQ ID NO: 4)
ATGACCCTGCTGGGCAGCGAGCACAGCCTGCTGATCAGATCCAAGTTCAGAAGCGTGCTGCAGC
TGAGACTGCAGCAGAGAAGAACACAGGAACAGCTGGCCAACCAGGGCATCATCCCCCCACTGAA
AAGACCTGCCGAGTTCCACGAGCAGAGAAAGCACCTGGACAGCGACAAGGCCAAGAACAGCCTG
AAGCGGAAGGCCCGGAATAGATGCAATAGCGCCGACCTGGTGAACATGCACATCCTGCAGGCTT
CCACCGCCGAGAGATCTATCCCTACAGCCCAGATGAAGCTGAAGAGAGCCAGACTGGCCGACGA
CCTGAATGAGAAGATTGCCCTGAGGCCTGGCCCCCTGGAACTGGTGGAAAAGAACATCCTGCCT
GTGGACAGCGCCGTGAAAGAGGCCATCAAGGGCAATCAGGTGTCCTTCAGCAAGAGCACCGACG
CCTTCGCCTTCGAGGAAGATTCTAGCTCTGACGGCCTGTCTCCTGATCAGACCAGATCTGAAGA
TCCTCAGAATAGCGCCGGCAGCCCTCCTGATGCCAAAGCCTCTGATACACCTTCTACCGGCAGC
CTGGGCACCAATCAGGATCTGGCCTCTGGCAGCGAGAACGACAGAAATGATAGCGCCAGCCAGC
CTAGCCACCAGTCTGATGCTGGAAAACAGGGCCTGGGCCCTCCTTCTACACCTATTGCTGTGCA
CGCCGCCGTGAAGTCTAAGAGCCTGGGCGACAGCAAGAACCGGCACAAGAAGCCTAAGGACCCC
AAGCCCAAAGTGAAGAAGCTGAAGTACCACCAGTACATCCCCCCCGACCAGAAGGCCGAAAAGT
CCCCTCCTCCTATGGATTCCGCCTACGCTAGACTGCTGCAACAGCAGCAGCTGTTCCTGCAGCT
CCAGATCCTGTCTCAACAACAGCAACAGCAGCAGCACCGGTTCAGCTATCTGGGAATGCACCAG
GCCCAGCTGAAAGAACCCAATGAGCAGATGGTCCGCAACCCCAATAGCAGCTCCACCCCTCTGA
GCAATACCCCCCTGAGCCCTGTGAAGAATAGCTTTTCTGGCCAGACCGGCGTGTCCAGCTTTAA
GCCTGGACCTCTGCCCCCCAACCTGGACGATCTGAAAGTGTCTGAACTGCGGCAGCAGCTGAGA
ATCAGAGGACTGCCTGTGTCTGGCACCAAGACCGCCCTGATGGATAGACTGAGGCCCTTTCAGG
ACTGCAGCGGCAACCCTGTGCCCAACTTTGGCGATATCACCACCGTGACCTTCCCCGTGACACC
CAACACCCTGCCTAATTACCAGAGCAGCAGCTCTACCAGCGCCCTGAGCAATGGCTTCTACCAC
TTTGGCAGCACAAGCAGCAGCCCTCCAATCAGCCCTGCCTCTTCTGATCTGTCTGTGGCCGGAA
GCCTGCCCGACACCTTTAATGATGCCAGCCCTAGCTTTGGCCTGCACCCTTCTCCAGTGCACGT
GTGCACAGAGGAATCCCTGATGTCTAGCCTGAATGGCGGCTCTGTGCCTTCTGAGCTGGATGGC
CTGGATTCCGAGAAGGACAAGATGCTGGTGGAAAAACAGAAAGTGATCAACGAGCTGACCTGGA
AGCTGCAGCAGGAACAGAGACAGGTGGAAGAACTGCGGATGCAGCTGCAGAAGCAGAAGCGGAA
CAACTGCTCCGAGAAGAAGCCTCTGCCATTCCTGGCCGCCAGCATTAAGCAGGAAGAGGCCGTG
TCAAGCTGCCCATTCGCCAGTCAGGTGCCAGTGAAGAGACAGAGCAGCTCCTCTGAATGTCACC
CTCCTGCTTGTGAAGCTGCCCAGCTGCAGCCTCTGGGAAATGCCCATTGTGTGGAAAGCAGCGA
CCAGACCAATGTGCTGAGCAGCACCTTCCTGAGCCCTCAGTGTTCTCCTCAGCATTCTCCCCTG
GGCGCTGTGAAGTCTCCACAGCACATTTCTCTGCCCCCTAGCCCCAACAACCCTCACTTTCTGC
CATCTAGTTCTGGCGCCCAGGGCGAGGGACATAGAGTGTCTAGTCCTATCAGCAGCCAGGTCTG
CACCGCTCAGAACTCTGGCGCTCATGATGGCCACCCTCCAAGCTTTAGCCCTCACTCTTCTAGC
CTGCACCCACCTTTTAGCGGAGCCCAGGCTGATTCTTCTCATGGCGCTGGCGGCAATCCTTGCC
CTAAGTCTCCTTGCGTGCAGCAGAAAATGGCCGGCCTGCACAGCTCTGACAAAGTGGGCCCTAA
GTTCAGCATCCCCAGCCCCACCTTTAGCAAGTCTAGCAGCGCCATCAGCGAAGTGACCCAGCCT
CCATCTTACGAGGATGCCGTGAAGCAGCAGATGACCAGATCCCAGCAGATGGACGAGCTGCTGG
ATGTGCTGATCGAGTCTGGCGAAATGCCTGCCGATGCCAGAGAGGATCACAGCTGTCTGCAGAA
GGTGCCCAAGATCCCCAGAAGCTCCAGATCTCCTACCGCCGTGCTGACAAAGCCTAGCGCCTCT
TTTGAGCAGGCCAGCAGCGGCAGCCAGATCCCTTTTGATCCTTACGCCACCGACAGCGACGAGC
ACCTGGAAGTGCTGCTGAATAGCCAGAGCCCTCTGGGCAAGATGAGCGACGTGACACTGCTGAA
GATCGGCAGCGAGGAACCCCACTTCGATGGCATCATGGATGGCTTTTCTGGAAAGGCCGCCGAG
GACCTGTTCAACGCCCACGAAATTCTGCCTGGCCCTCTGAGCCCTATGCAGACCCAGTTTAGCC
CTAGCAGCGTGGACTCTAATGGCCTGCAGCTGTCCTTTACCGAGAGCCCCTGGGAGACAATGGA
ATGGCTGGACCTGACCCCCCCTAATAGCACACCTGGATTTTCTGCCCTGACCACCAGCAGCCCC
TCTATCTTCAATATCGACTTCCTGGACGTGACCGACCTGAACCTGAACAGCAGCATGGACCTGC
ATCTGCAGCAGTGGTGA

In particular embodiments, part or all of SEQ ID NOs: 3 and/or 4 is utilized in methods of the disclosure. In specific embodiments, a polynucleotide having a specific sequence identity with respect to SEQ ID NOs: 3 and/or 4 is utilized in methods of the disclosure. In specific cases, a functional fragment of SEQ ID NOs: 3 and/or 4 is employed, and the term “functional fragment” as used herein refers to a polynucleotide that encodes a polypeptide having the activity of being able to convert fibroblasts to endothelial cells or endothelial-like cells. In specific cases, the fragment has a length of at least about or no more than about 2900, 2800, 2700, 2600, 2500, 2400, 2300, 2200, 2100, 2000, 1900,1800, 1700,1600, 1500,1400, 1375, 1350, 1325, 1300, 1275, 1250, 1225, 1200, 1175, 1150, 1125, 1100, 1075, 1050, 1025, 1000, 975, 950, 925, 900, 875, 850, 825, 800, 775, 750, 725, 700, 675, 650, 625, 600, 575, 550, 525, 500, 475, 450, 425, 400, 375, 350, 325, 300, 275, 250, 225, 200, 175, 150, 125, or 100 contiguous nucleotides of SEQ ID NOs: 3 and/or 4. In addition, the fragment may have sequence identity with the corresponding region in SEQ ID NOs: 3 and/or 4 of at least, or exactly, 100, 99, 98, 97, 96, 95, 94, 93, 92, 91, 90, 89, 88, 87, 86, 85, 80, 75, or 70% identity. A polynucleotide having certain sequence identity to SEQ ID NOs: 3 and/or 4 may be used, including at least, or exactly 100, 99, 98, 97, 96, 95, 94, 93, 92, 91, 90, 89, 88, 87, 86, 85, 80, 75, or 70% identity to SEQ ID NOs: 3 and/or 4.

In some embodiments, a Hand2 and/or myocardin polypeptide is delivered to an individual in need thereof, whether it be in the form of being on a vector, associated with a carrier, within a cell (including in a cell on a vector), and so forth. In specific embodiments, the Hand2 and/or myocardin polypeptide is a mammalian Hand2 and/or myocardin polypeptide, including human, mouse, rat, and so forth. In particular embodiments, one example of a Hand2 polypeptide sequence is and/or is comprised in SEQ ID NO: 5. In particular embodiments, one example of a myocardin polypeptide sequence is and/or is comprised in SEQ ID NO: 6.

(SEQ ID NO: 5)
MSLVGGFPHHPVVHHEGYPFAAAAAAAAAAAASRCSHEENPYFHGWLIGH
PEMSPPDYSMALSYSPEYASGAAGLDHSHYGGVPPGAGPPGLGGPRPVKR
RGTANRKERRRTQSINSAFAELRECIPNVPADTKLSKIKTLRLATSYIAY
LMDLLAKDDQNGEAEAFKAEIKKTDVKEEKRKKELNEILKSTVSSNDKKT
KGRTGWPQHVWALELKQGSG
(SEQ ID NO: 6)
MTLLGSEHSLLIRSKFRSVLQLRLQQRRTQEQLANQGIIPPLKRPAEFHE
QRKHLDSDKAKNSLKRKARNRCNSADLVNMHILQASTAERSIPTAQMKLK
RARLADDLNEKIALRPGPLELVEKNILPVDSAVKEAIKGNQVSFSKSTDA
FAFEEDSSSDGLSPDQTRSEDPQNSAGSPPDAKASDTPSTGSLGTNQDLA
SGSENDRNDSASQPSHQSDAGKQGLGPPSTPIAVHAAVKSKSLGDSKNRH
KKPKDPKPKVKKLKYHQYIPPDQKAEKSPPPMDSAYARLLQQQQLFLQLQ
ILSQQQQQQQHRFSYLGMHQAQLKEPNEQMVRNPNSSSTPLSNTPLSPVK
NSFSGQTGVSSFKPGPLPPNLDDLKVSELRQQLRIRGLPVSGTKTALMDR
LRPFQDCSGNPVPNFGDITTVTFPVTPNTLPNYQSSSSTSALSNGFYHFG
STSSSPPISPASSDLSVAGSLPDTFNDASPSFGLHPSPVHVCTEESLMSS
LNGGSVPSELDGLDSEKDKMLVEKQKVINELTWKLQQEQRQVEELRMQLQ
KQKRNNCSEKKPLPFLAASIKQEEAVSSCPFASQVPVKRQSSSSECHPPA
CEAAQLQPLGNAHCVESSDQTNVLSSTFLSPQCSPQHSPLGAVKSPQHIS
LPPSPNNPHFLPSSSGAQGEGHRVSSPISSQVCTAQNSGAHDGHPPSFSP
HSSSLHPPFSGAQADSSHGAGGNPCPKSPCVQQKMAGLHSSDKVGPKFSI
PSPTFSKSSSAISEVTQPPSYEDAVKQQMTRSQQMDELLDVLIESGEMPA
DAREDHSCLQKVPKIPRSSRSPTAVLTKPSASFEQASSGSQIPFDPYATD
SDEHLEVLLNSQSPLGKMSDVILLKIGSEEPHFDGIMDGFSGKAAEDLFN
AHEILPGPLSPMQTQFSPSSVDSNGLQLSFTESPWETMEWLDLTPPNSTP
GFSALTTSSPSIFNIDFLDVTDLNLNSSMDLHLQQW

In particular embodiments, part or all of SEQ ID NOs: 5 and/or 6 is utilized in methods of the disclosure. In specific embodiments, a polypeptide having a specific sequence identity with respect to SEQ ID NOs: 5 and/or 6 is utilized in methods of the disclosure. In specific cases, a functional fragment of SEQ ID NOs: 5 and/or 6 is employed, and the term “functional fragment” as used herein refers to a polypeptide having the activity of being able to convert fibroblasts to endothelial cells or endothelial-like cells. In specific cases, the fragment has a length of at least about or no more than about 975, 950, 925, 900, 875, 850, 825, 800, 775, 750, 725, 700, 675, 650, 625, 600, 575, 550, 525, 500, 475, 450, 425, 400, 375, 350, 325, 300, 275, 250, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, or 20 contiguous amino acids SEQ ID NOs: 5 and/or 6.

### B. Chromatin Destabilizing Agents

In particular embodiments, one or more chromatin destabilizing agents are utilized with p63-TID (or a functional fragment and/or a functional derivative thereof). The one or more chromatin destabilizing agents may be provided to an individual at the same time as the p63-TID (or a functional fragment and/or a functional derivative thereof), although in specific embodiments the one or more chromatin destabilizing agents are utilized before or after p63-TID (or a functional fragment and/or a functional derivative thereof).

Although one could use standard methods to test whether or not a certain compound would be effective as a chromatin destabilizing agent, in specific embodiments the chromatin destabilizing agent is Oct4, DZNep, Sall4, SOX2, KLF4, MYC, SB431542, PD0325901, Parnate. CHIR99021, A-83-01. NaB, PS48, Forskolin (FSK), 2-methyl-5-hydroxytryptamine (2-Me-5HT), D4476, VPA,CHIR99021 (CHIR), 616452, Tranylcypromine, Prostaglandin E2, Rolipram, 3-deazaneplanocin A (DZNep), 5-Azacytidine, sodium butyrate, RG108 or a combination thereof.

### V. Nucleic Acids, Generally

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length, in at least some cases.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

As used herein “wild-type” refers to the naturally occurring sequence of a nucleic acid at a genetic locus in the genome of an organism, or a sequence transcribed or translated from such a nucleic acid. Thus, the term “wild-type” also may refer to an amino acid sequence encoded by a nucleic acid. As a genetic locus may have more than one sequence or alleles in a population of individuals, the term “wild-type” encompasses all such naturally occurring allele(s). As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

The present disclosure also concerns the isolation or creation of a recombinant construct or a recombinant host cell through the application of recombinant nucleic acid technology known to those of skill in the art or as described herein. A recombinant construct or host cell may comprise a nucleic acid, and may express a protein, peptide or peptide, or at least one biologically functional equivalent thereof.

Herein, in certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

“Isolated substantially away from other coding sequences” means that the gene of interest, or fragment thereof, forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

The nucleic acid(s) of the present disclosure, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared that include a contiguous stretch of nucleotides identical to or complementary (at least in part) to p63. A nucleic acid construct may be about 3, about 5, about 8, about 10 to about 14, or about 15, about 20, about 30, about 40, about 50, about 100, about 115, about 200, about 500, about 600, or about 650 nucleotides in length, as well as constructs of greater size, up to and including vector sizes (including all intermediate lengths and intermediate ranges. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 600, about 601, about 605, about 610, etc. Non-limiting examples of intermediate ranges include about 3 to about 32, about 150 to about 750, etc.

In certain embodiments, the nucleic acid construct is a recombinant vector. In particular embodiments, the disclosure concerns one or more recombinant vector(s) comprising nucleic acid sequences that encode an Hand2 or myocardin protein, polypeptide or peptide. In particular aspects, the recombinant vectors are DNA vectors.

The term “biologically functional equivalent” is well understood in the art and is further defined in detail herein. Accordingly, a sequence that has 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of amino acids that are identical or functionally equivalent to the amino acids encoded by the Hand2 and myocardin nucleic acids, respectively, provided the biological activity of the protein, polypeptide or peptide is maintained.

In certain other embodiments, the disclosure concerns at least one recombinant vector that includes within its sequence a nucleic acid sequence that expresses p63-TID (or a functional fragment and/or afunctional derivative thereof). In specific embodiments, the disclosure concerns at least one recombinant vector that includes within its sequence a nucleic acid sequence that expresses Hand2 nucleic acid. In another embodiment, there is at least one recombinant vector that includes within its sequence a nucleic acid sequence that expresses myocardin nucleic acid.

The term “functionally equivalent codon” is used herein to refer to codons that encode the same amino acid, such as the six codons for arginine and serine, and also refers to codons that encode biologically equivalent amino acids. Codon usage for various organisms and organelles can be found in the literature. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of the noted GENBANK® sequences disclosed herein are encompassed in the disclosure.

Recombinant vectors and isolated nucleic acid segments may therefore variously include Hand2 or myocardin coding regions themselves, coding regions bearing selected alterations or modifications in the basic coding region, and they may encode larger polypeptides or peptides that nevertheless include such coding regions or may encode biologically functional equivalent proteins, polypeptide or peptides that have variant amino acids sequences.

The nucleic acids of the present disclosure may encompass biologically functional equivalent coding sequences for Hand2 or myocardin proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine protein, polypeptide or peptide activity at the molecular level.

### A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like.

A nucleobase may be comprised in a nucleoside or nucleotide, using any chemical or natural synthesis method described herein or known to one of ordinary skill in the art.

### B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

### C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

### D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moiety which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moiety replacing phosphodiester backbone moiety used for improved nuclease resistance, cellular uptake and regulating RNA expression; U.S. Pat. No. 5,858,988 which describes hydrophobic carrier agent attached to the 2′-O position of oligonuceotides to enhanced their membrane permeability and stability; U.S. Pat. No. 5,214,136 which describes olignucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

### E. Polyether and Peptide Nucleic Acids

In certain embodiments, it is contemplated that a nucleic acid comprising a derivative or analog of a nucleoside or nucleotide may be used in the methods and compositions of the disclosure. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (see e.g., Egholm et al., 1993; PCT/EP/01219). A peptide nucleic acid generally comprises one or more nucleotides or nucleosides that comprise a nucleobase moiety, a nucleobase linker moiety that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5,891,625. Other modifications and uses of nucleic acid analogs are known in the art, and are encompassed herein. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. application Ser. No. 117,363 describes several alkylamino moeities used to enhance cellular uptake of a PNA. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

### F. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present disclosure, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

### G. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present disclosure concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

### H. Nucleic Acid Segments

In certain embodiments, the nucleic acid is a nucleic acid segment. As used herein, the term “nucleic acid segment,” are smaller fragments of a nucleic acid, such as for non-limiting example, those that encode only part of the peptide or polypeptide sequence. Thus, a “nucleic acid segment” may comprise any part of a gene sequence, of from about 2 nucleotides to the full length of the peptide or polypeptide encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

### VI. Nucleic Acid-Based Expression Systems

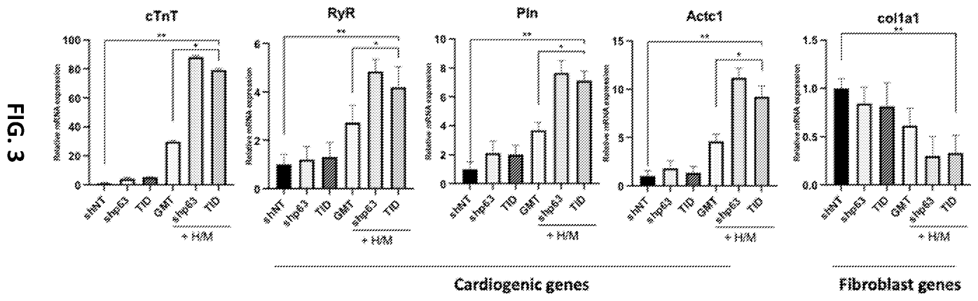

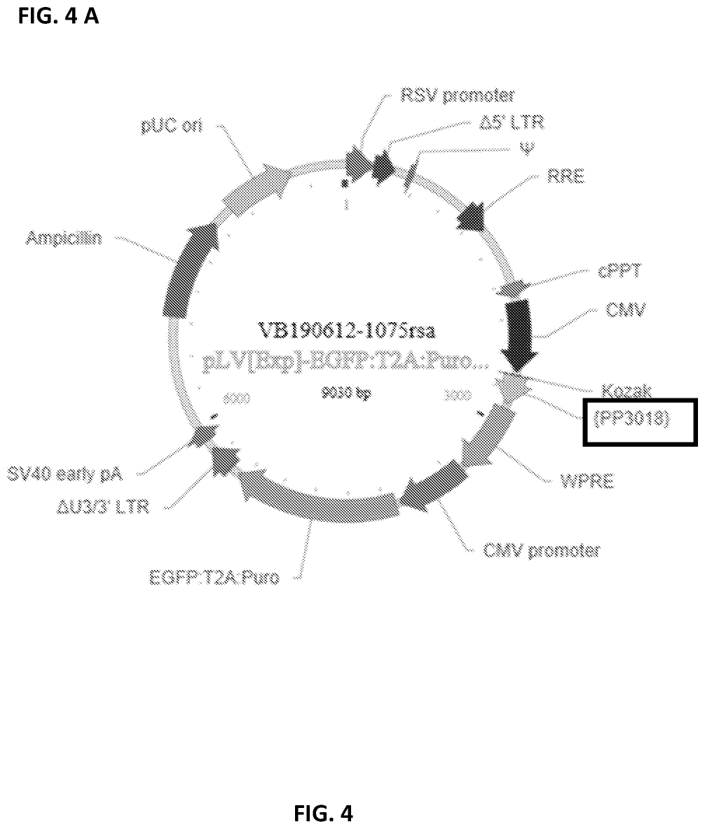

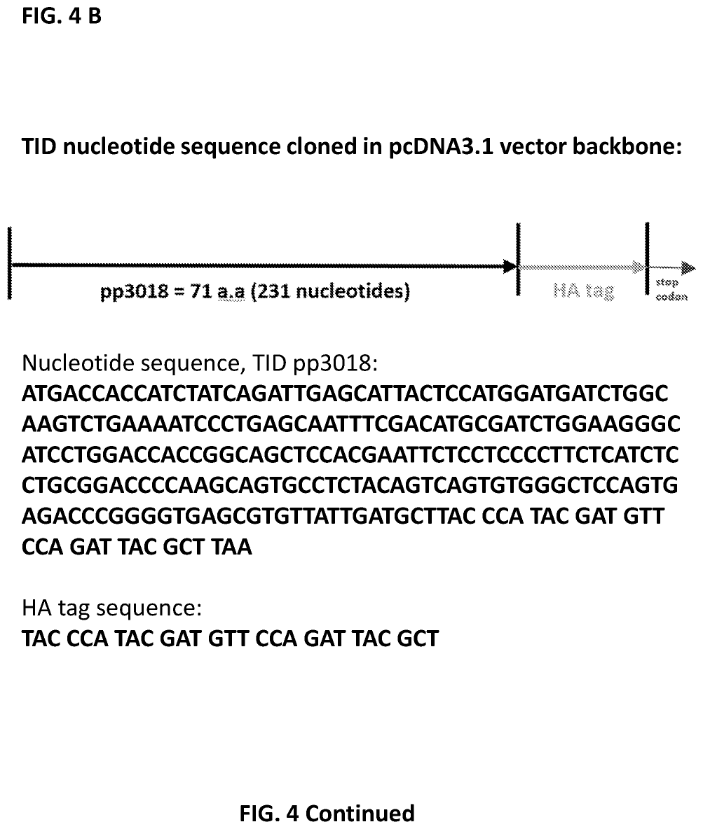

In particular embodiments of the disclosure, p63-TID (or a functional fragment and/or a functional derivative thereof) (such as polypeptide or nucleic acids), in some cases one or more cardiac cell reprogramming factors (such as Hand2 and/or myocardin), and in some cases one or more destabilizing agents and/or anti-fibrotic agents and/or angiogenic factors are provided in nucleic acid form to an individual in need thereof. Although in some cases the nucleic acids are not comprised on a vector, in particular embodiments the nucleic acids are present on one or more vectors. In particular embodiments, the different nucleic acids are present on the same vector, whereas in other cases they are present on two or three separate vectors. The vectors may be viral or non-viral in nature. FIGS. 4 A-B provide an illustration of embodiments of vectors for use in methods of the present disclosure.

The vectors utilized in the embodiments of the disclosure may have one or more means for targeted delivery to cardiac tissue and/or targeted expression in certain cells. In some cases the vector(s) are provided to the individual with localized delivery to the heart, whereas in other cases the vectors are provided systemically to the individual with a means for targeted delivery to cardiac tissue and/or targeted expression in certain cells, such as cardiac fibroblasts, for example. In certain embodiments, the p63-TID (or a functional fragment and/or a functional derivative thereof) and one or both of cardiac cell reprogramming factors (such as Hand2 and myocardin) polynucleotides are on the same molecule, although in some embodiments the p63-TID (or a functional fragment and/or a functional derivative thereof) and one or both of cardiac cell reprogramming factors polynucleotides are on different molecules. When the p63-TID (or a functional fragment and/or a functional derivative thereof) and one or both of cardiac cell reprogramming factors are expressed from the same polynucleotide, they may have the same or different regulatory regions for their expression. In specific embodiments, the p63-TID (or a functional fragment and/or a functional derivative thereof) and one or more chromatin destabilizing agent polynucleotides are on the same or different molecules.

In particular embodiments, an expression vector for use in the disclosure may comprise one or more suitable restriction enzyme digestion sequences, start codons, stop codons, nuclear localization signals, protease cutting codons, selectable markers, origins of replication, regulatory regions, multiple cloning sites, and a combination thereof. Such moieties may be positioned in the expression vector in any suitable order.

### A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

### 1. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

In embodiments of the disclosure, a CMV promoter or a tissue-specific promoter may be employed. The tissue-specific promoter may be a cardiac tissue specific promoter. Examples of cardiac tissue specific promoters include but are not limited to ventricle-specific myosin light chain-2 (mlc-2v); and/or alpha-myosin heavy chain (a-MHC). In specific embodiments, a fibroblast-specific promoter is employed. Examples of fibroblast-specific promoters include but are not limited to Fsp1 and/or periostin.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the P-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

### 2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the disclosure, the use of internal ribosome entry sites (IRES) elements are used to create multigene, or polycistronic, messages. IRES elements are able to bypass the ribosome scanning model of 5′ methylated Cap dependent translation and begin translation at internal sites (see e.g., Pelletier and Sonenberg, 1988). IRES elements from two members of the picornavirus family (polio and encephalomyocarditis) have been described (Pelletier and Sonenberg, 1988), as well an IRES from a mammalian message (see e.g., Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### 3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### 4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

### 5. Termination Signals

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### 6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the disclosure, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

### 7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

### 8. Selectable and Screenable Markers

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### 9. Additional Features

In some embodiments, vectors and/or nucleic acid constructs of the present disclosure may comprise a 2A element or sequence. In some embodiments, vectors and/or nucleic acid constructs of the present disclosure may include one or more cloning sites. In some such embodiments, cloning sites may not be fully removed prior to manufacturing for administration to a subject. In some embodiments, cloning sites may have functional roles including as linker sequences, or as portions of a Kozak site. As will be appreciated by those skilled in the art, cloning sites may vary significantly in primary sequence while retaining their desired function.

In some embodiments, a 2A element is a T2A, P2A, E2A, and/or F2A element. In some embodiments, a 2A sequence may comprise an optional 5′ linker sequence, such as but not limited to GSG (Glycine, Serine, Glycine).

SEQ ID NO: 7
Exemplary T2A amino acid sequence
EGRGSLLTCGDVEENPGP
SEQ ID NO: 8
Exemplary P2A amino acid sequence
ATNFSLLKQAGDVEENPGP
SEQ ID NO: 9
Exemplary E2A amino acid sequence
QCTNYALLKLAGDVESNPGP
SEQ ID NO: 10
Exemplary F2A amino acid sequence
VKQTLNFDLLKLAGDVESNPGP
SEQ ID NO: 11
Exemplary P2A nucleotide sequence
GCCACAAACTTCAGCCTGCTGAAACAGGCTGGCGACGTGGAAGAGAACCC
TGGCCCTTCTAGA

### B. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Genomic integrated plasmids, such as piggybac or sleeping beauty transposon gene delivery plasmids, may be employed for long term transgenic expression of a nucleic acid in heart or other organ.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with P-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

### C. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present disclosure are described below.

### 1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

### 2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in embodiments of the present disclosure as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference. In certain embodiments, an AAV (e.g., including rAAV vectors) vector is specific for and/or has increased specificity for muscle cells and/or myocardial cells when compared to an appropriate control vector.

### 3. Retroviral Vectors

Retroviruses have promise as delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

### 4. Other Viral Vectors

Other viral vectors may be employed as vaccine constructs in the present disclosure. Vectors derived from viruses such as vaccinia virus (see e.g., Ridgeway, 1988; Baichwal and Sugden, 1986; Coupar et al., 1988), sindbis virus, cytomegalovirus and herpes simplex virus may be employed. They offer several attractive features for various mammalian cells (see e.g., Friedmann, 1989; Ridgeway, 1988; Baichwal and Sugden, 1986; Coupar et al., 1988; Horwich et al., 1990).

### D. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (see e.g., Roux et al., 1989). Using antibodies against major histocompatibility complex class I and class II antigens, they demonstrated the infection of a variety of human cells that bore those surface antigens with an ecotropic virus in vitro (see e.g., Roux et al., 1989).

### E. Vector Delivery and Cell Transformation

Suitable methods for nucleic acid delivery for transformation of an organelle, a cell, a tissue or an organism for use with the current disclosure are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (see e.g., Wilson et al., 1989, Nabel et al, 1989), by injection (see e.g., U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (see e.g., Harlan and Weintraub, 1985; U.S. Pat. No. 5,789,215, incorporated herein by reference); by electroporation (see e.g., U.S. Pat. No. 5,384,253, incorporated herein by reference; Tur-Kaspa et al., 1986; Potter et al., 1984); by calcium phosphate precipitation (see e.g., Graham and Van Der Eb, 1973; Chen and Okayama, 1987; Rippe et al., 1990); by using DEAE-dextran followed by polyethylene glycol (see e.g., Gopal, 1985); by direct sonic loading (see e.g., Fechheimer et al., 1987); by liposome mediated transfection (see e.g., Nicolau and Sene, 1982; Fraley et al., 1979; Nicolau et al., 1987; Wong et al., 1980; Kaneda et al., 1989; Kato et al., 1991) and receptor-mediated transfection (see e.g., Wu and Wu, 1987; Wu and Wu, 1988); by microprojectile bombardment (see e.g., PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (see e.g., Kaeppler et al., 1990; U.S. Pat. Nos. 5,302,523 and 5,464,765, each incorporated herein by reference); by Agrobacterium-mediated transformation (see e.g., U.S. Pat. Nos. 5,591,616 and 5,563,055, each incorporated herein by reference); by PEG-mediated transformation of protoplasts (see e.g., Omirulleh et al., 1993; U.S. Pat. Nos. 4,684,611 and 4,952,500, each incorporated herein by reference); by desiccation/inhibition-mediated DNA uptake (see e.g., Potrykus et al., 1985), and any combination of such methods. Through the application of techniques such as these, organelle(s), cell(s), tissue(s) or organism(s) may be stably or transiently transformed.

### 1. Ex Vivo Transformation

Methods for transfecting vascular cells and tissues removed from an organism in an ex vivo setting are known to those of skill in the art. For example, canine endothelial cells have been genetically altered by retroviral gene transfer in vitro and transplanted into a canine (see e.g., Wilson et al., 1989). In another example, Yucatan minipig endothelial cells were transfected by retrovirus in vitro and transplanted into an artery using a double-balloon catheter (see e.g., Nabel et al., 1989). Thus, it is contemplated that cells or tissues may be removed and transfected ex vivo using the nucleic acids of the present disclosure. In particular aspects, the transplanted cells or tissues may be placed into an organism. In preferred facets, a nucleic acid is expressed in the transplanted cells or tissues.

### 2. Injection

In certain embodiments, a nucleic acid may be delivered to an organelle, a cell, a tissue or an organism via one or more injections (i.e., a needle injection), such as, for example, intramyocardially, endocardially, epicardially, intra-coronarily, by direct injection or intracoronary injection, subcutaneously, intradermally, intramuscularly, intervenously, intraperitoneally, etc. In specific embodiments, p63-TID is delivered directly to the heart by injection. Methods of injection are well known to those of ordinary skill in the art (e.g., injection of a composition comprising a saline solution). Further embodiments of the present disclosure include the introduction of a nucleic acid by direct microinjection.

### 3. Electroporation

In certain embodiments of the present disclosure, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (see e.g., U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (see e.g., Potter et al., 1984), and rat hepatocytes have been transfected with the chloramphenicol acetyltransferase gene (see e.g., Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (see e.g., U.S. Pat. No. 5,384,253; Rhodes et al., 1995; D'Halluin et al., 1992), wheat (see e.g., Zhou et al., 1993), tomato (see e.g., Hou and Lin, 1996), soybean (see e.g., Christou et al., 1987) and tobacco (see e.g., Lee et al., 1989).

One also may employ protoplasts for electroporation transformation of plant cells (see e.g., Bates, 1994; Lazzeri, 1995). For example, the generation of transgenic soybean plants by electroporation of cotyledon-derived protoplasts is described by Dhir and Widholm in International Patent Application No. WO 9217598, incorporated herein by reference. Other examples of species for which protoplast transformation has been described include barley (see e.g., Lazerri, 1995), sorghum (see e.g., Battraw et al., 1991), maize (see e.g., Bhattacharjee et al., 1997), wheat (see e.g., He et al., 1994) and tomato (see e.g., Tsukada, 1989).

### 4. Calcium Phosphate

In other embodiments of the present disclosure, a nucleic acid is introduced to the cells using calcium phosphate precipitation. Human KB cells have been transfected with adenovirus 5 DNA (see e.g., Graham and Van Der Eb, 1973) using this technique. Also in this manner, mouse L(A9), mouse C127, CHO, CV-1, BHK, NIH3T3 and HeLa cells were transfected with a neomycin marker gene (see e.g., Chen and Okayama, 1987), and rat hepatocytes were transfected with a variety of marker genes (see e.g., Rippe et al., 1990).

### 5. DEAE-Dextran

In another embodiment, a nucleic acid is delivered into a cell using DEAE-dextran followed by polyethylene glycol. In this manner, reporter plasmids were introduced into mouse myeloma and erythroleukemia cells (see e.g., Gopal, 1985).

### 6. Sonication Loading

Additional embodiments of the present disclosure include the introduction of a nucleic acid by direct sonic loading. LTK-fibroblasts have been transfected with the thymidine kinase gene by sonication loading (see e.g., Fechheimer et al., 1987).

### 7. Liposome-Mediated Transfection

In a further embodiment of the disclosure, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (see e.g., Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (see e.g., Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (see e.g., Wong et al., 1980).

In certain embodiments of the disclosure, a liposome may be complexed with a hemagglutinating virus (HVJ). This has been shown to facilitate fusion with the cell membrane and promote cell entry of liposome-encapsulated DNA (see e.g., Kaneda et al., 1989). In other embodiments, a liposome may be complexed or employed in conjunction with nuclear non-histone chromosomal proteins (HMG-1) (see e.g., Kato et al., 1991). In yet further embodiments, a liposome may be complexed or employed in conjunction with both HVJ and HMG-1. In other embodiments, a delivery vehicle may comprise a ligand and a liposome.

### 8. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present disclosure.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (see e.g., Wu and Wu, 1987; Wagner et al., 1990; Perales et al., 1994; Myers, EPO 0273085), which establishes the operability of the technique. Specific delivery in the context of another mammalian cell type has been described (see e.g., Wu and Wu, 1993; incorporated herein by reference). In certain aspects of the present disclosure, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (see e.g., Nicolau et al., 1987). It is contemplated that the tissue-specific transforming constructs of the present disclosure can be specifically delivered into a target cell in a similar manner.

### 9. Microprojectile Bombardment

Microprojectile bombardment techniques can be used to introduce a nucleic acid into at least one, organelle, cell, tissue or organism (see e.g., U.S. Pat. Nos. 5,550,318; 5,538,880; 5,610,042; and PCT Application WO 94/09699; each of which is incorporated herein by reference). This method depends on the ability to accelerate DNA-coated microprojectiles to a high velocity allowing them to pierce cell membranes and enter cells without killing them (see e.g., Klein et al., 1987). There are a wide variety of microprojectile bombardment techniques known in the art, many of which are applicable to the disclosure.

Microprojectile bombardment may be used to transform various cell(s), tissue(s) or organism(s), such as for example any plant species. Examples of species which have been transformed by microprojectile bombardment include monocot species such as maize (see e.g., PCT Application WO 95/06128), barley (see e.g., Ritala et al., 1994; Hensgens et al., 1993), wheat (see e.g., U.S. Pat. No. 5,563,055, incorporated herein by reference), rice (see e.g., Hensgens et al., 1993), oat (see e.g., Torbet et al., 1995; Torbet et al., 1998), rye (see e.g., Hensgens et al., 1993), sugarcane (see e.g., Bower et al., 1992), and sorghum (see e.g., Casas et al., 1993; Hagio et al., 1991); as well as a number of dicots including tobacco (see e.g., Tomes et al., 1990; Buising and Benbow, 1994), soybean (see e.g., U.S. Pat. No. 5,322,783, incorporated herein by reference), sunflower (see e.g., Knittel et al. 1994), peanut (see e.g., Singsit et al., 1997), cotton (see e.g., McCabe and Martinell, 1993), tomato (see e.g., VanEck et al. 1995), and legumes in general (see e.g., U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (see e.g., Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the microprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

### VII. Proteins, Polypeptides, and Peptides

In some cases, embodiments may utilize p63-TID as a polypeptide and optionally may utilize one or more purified cardiac cell programming factors, such as Hand2, myocardin, Gata4, Mef2c, or Tbx5 proteins, polypeptides, or peptides, or one or more chromatin destabilizing agent proteins, polypeptides, or peptides, or other proteins, polypeptides, or peptides, and this may be done in addition to or alternative to utilizing the respective nucleic acid form. The term “purified proteins, polypeptides, or peptides” as used herein, is intended to refer to an proteinaceous composition, isolatable from mammalian cells or recombinant host cells, wherein the at least one protein, polypeptide, or peptide is purified to any degree relative to its naturally-obtainable state, i.e., relative to its purity within a cellular extract. A purified protein, polypeptide, or peptide therefore also refers to a wild-type or mutant protein, polypeptide, or peptide free from the environment in which it naturally occurs.

The nucleotide and protein, polypeptide and peptide sequences for various genes have been previously disclosed, and may be found at computerized databases known to those of ordinary skill in the art. One such database is the National Center for Biotechnology Information's GENBANK® and GENPEPT® databases. The coding regions for these known genes may be amplified and/or expressed using the techniques disclosed herein or by any technique that would be known to those of ordinary skill in the art. Additionally, peptide sequences may be synthesized by methods known to those of ordinary skill in the art, such as peptide synthesis using automated peptide synthesis machines, such as those available from Applied Biosystems (Foster City, CA).

Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present disclosure, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of a specific fusion protein using a specific binding partner. Such purification methods are routine in the art. As the present disclosure provides DNA sequences for the specific proteins, any fusion protein purification method can now be practiced. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. However, given many DNA and proteins are known, or may be identified and amplified using the methods described herein, any purification method can now be employed.

Although considered for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

Methods exhibiting a lower degree of relative purification may have advantages in total recovery of protein product, or in maintaining the activity of an expressed protein. Inactive products also have utility in certain embodiments, such as, e.g., in determining antigenicity via antibody generation.

### VIII. Host Cells

Although in some embodiments the nucleic acids of the disclosure are provided directly to cardiac tissue and are taken up by cells in the tissue, in some embodiments the nucleic acids are first generated and manipulated in cells ex vivo, such as by employing routine recombinant technology methods.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs or DNAs (as an active agent) or polypeptides (as an active agent), which could then be expressed in host cells transfected with the single vector.

A tissue may comprise a host cell or cells to be transformed with a polynucleotide or nucleic acid encoding p63-TID (or a functional fragment and/or a functional derivative thereof), one or more cardiac cell reprogramming factors, and/or one or more chromatin destabilizing agents. In specific embodiments, a cell may harbor a polynucleotide encoding p63-TID (or a functional fragment and/or a functional derivative thereof), Hand2, and/or myocardin. The tissue may be part or separated from an organism. In certain embodiments, a tissue may comprise, but is not limited to, myocytes, adipocytes, alveolar, ameloblasts, axon, basal cells, blood (e.g., lymphocytes), blood vessel, bone, bone marrow, brain, breast, cardiac, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, and so forth.

In certain embodiments, the host cell or tissue may be comprised in at least one organism. In certain embodiments, the organism may be, but is not limited to, a prokaryote (e.g., a eubacteria, an archaea) or an eukaryote, as would be understood by one of ordinary skill in the art (see, for example, webpage http://phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5a, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### IX. Combination Therapy

In certain cases, the therapy of the present disclosure is utilized in conjunction with one or more other therapies for a cardiac medical condition. p63-TID (or a functional fragment and/or a functional derivative thereof) may be used in conjunction with one or more cardiac cell reprogramming factors and/or in conjunction with one or more chromatin destabilizing agents and/or with one or more anti-fibrotic agents or angiogenic factors. In specific embodiments, p63-TID (or a functional fragment and/or a functional derivative thereof) is used in combination with Hand2 and or myocardin gene therapy, although it may also be used in combination with other genes or gene products, including, Gata4, Mef2c, Tbx5, miR-133, miR-1, Oct4, Klf4, c-myc, Sox2, Mesp1, Brachyury, Nkx2.5, ETS2, ESRRG, Mrtf-A, MyoD, and/or ZFPM2 (in nucleic acid or polypeptide or peptide form, in specific embodiments). The one or more other therapies may be directly or indirectly related to the cardiac medical condition (examples of indirectly related therapies include those for pain or infection). In specific embodiments, the additional therapy related to the cardiac medical condition is drug therapy, surgery, ventricular assisted device (VAD) implantation, video assisted thoracotomy (VAT), coronary bypass, or a combination thereof.

In specific embodiments, one or more agents that prevent fibrosis and/or enhance or promote angiogenesis may be used as adjuncts to embodiments of the disclosure. They may be provided to an individual in a localized region of the heart, including a region that has tissue damage, loss of cardiomyocyte, scar tissue, and so forth, or they may be provided systemically. The one or more agents may be any composition suitable to facilitate angiogenesis in the desired region. In specific embodiments, the agent may be a protein, peptide, small molecule, nucleic acid, and so forth. Embodiments such as those described in US2003/0103943 or US2001/0041679 may be employed in conjunction with the methods of the disclosure. Embodiments such as those described in US2018/0066252A1 or US2022/0143142A1 (each of which are incorporated herein in their entirety for the purposes described herein) may be employed in conjunction with the methods of the disclosure. Specific embodiments include fibroblast growth factor (FGF); vascular endothelial growth factor (VEGF); Ets variant 2 (ETV2); angiopoietins, Ang1 and Ang2; matrix metalloproteinase (MMP); Delta-like ligand 4 (DII4); or peptides thereof; or combinations thereof. ITD-1 is a small molecule that inhibits TGF-beta and thus, fibrosis and cardiac remodeling (Willems E, Cabral-Teixeira J, Schade D, et al. Cell Stem Cell. 2012. pp. 242-252.), and it may be utilized.

In specific embodiments, an agent that enhances angiogenesis is VEGF. In specific embodiments, an agent that enhances angiogenesis is ETV2. In certain embodiments, VEGF and/or ETV2 are administered using any method described herein, such as but not limited to proteins/peptides, vectors, plasmid vectors, and/or viral vectors. In certain embodiments VEGF and/or ETV2 are administered using an adenoviral vector. In certain embodiments, VEGF and/or ETV2 are administered simultaneously with p63-TID and H/M. In certain embodiments, VEGF and/or ETV2 are administered prior to administration of p63-TID and H/M. In certain embodiments, VEGF and/or ETV2 administration with p63-TID and H/M results in superior cardiac cell reprogramming when compared to VEGF and/or ETV2 administration with control, GMT, and/or GMTd treatments.

In certain embodiments, an exemplary ETV2 polynucleotide sequence is and/or is comprised within the GENBANK® Accession No. NM_001300974 (SEQ ID NO: 12):

(SEQ ID NO: 12)
TTCCTGTTGCAGATAAGCCCAGCTTAGCCCAGCTGACCCCAGACCCTCTC
CCCTCACTCCCCCCATGTCGCAGGATCGAGACCCTGAGGCAGACAGCCCG
TTCACCAAGCCCCCCGCCCCGCCCCCATCACCCCGTAAACTTCTCCCAGC
CTCCGCCCTGCCCTCACCCAGCCCGCTGTTCCCCAAGCCTCGCTCCAAGC
CCACGCCACCCCTGCAGCAGGGCAGCCCCAGAGGCCAGCACCTATCCCCG
AGGCTGGGGTCGAGGCTCGGCCCCGCCCCTGCCTCTGCAACTTGAGCCTG
GCTGCGACCCCTGCTCTGACGTCTCGGAAAATTCCCCCTTGCCCAGGCCC
TTGGGGGAGGGGGTGCATGGTATGAAATGGGGCTGAGACCCCCGGCTGGG
GGCAGAGGAACCCGCCAGAGAAGGAGCCAAATTAGGCTTCTGTTTCCCTG
ATCTGGCACTCCAAGGGGACACGCCGACAGCGACAGCAGAGACATGCTGG
AAAGGTACAAGCTCATCCCTGGCAAGCTTCCCACAGCTGGACTGGGGCTC
CGCGTTACTGCACCCAGAAGTTCCATGGGGGGCGGAGCCCGACTCTCAGG
CTCTTCCGTGGTCCGGGGACTGGACAGACATGGCGTGCACAGCCTGGGAC
TCTTGGAGCGGCGCCTCGCAGACCCTGGGCCCCGCCCCTCTCGGCCCGGG
CCCCATCCCCGCCGCCGGCTCCGAAGGCGCCGCGGGCCAGAACTGCGTCC
CCGTGGCGGGAGAGGCCACCTCGTGGTCGCGCGCCCAGGCCGCCGGGAGC
AACACCAGCTGGGACTGTTCTGTGGGGCCCGACGGCGATACCTACTGGGG
CAGTGGCCTGGGGGGGGAGCCGCGCACGGACTGTACCATTTCGTGGGGCG
GGCCCGCGGGCCCGGACTGTACCACCTCCTGGAACCCGGGGCTGCATGCG
GGTGGCACCACCTCTTTGAAGCGGTACCAGAGCTCAGCTCTCACCGTTTG
CTCCGAACCGAGCCCGCAGTCGGACCGTGCCAGTTTGGCTCGATGCCCCA
AAACTAACCACCGAGGTCCCATTCAGCTGTGGCAGTTCCTCCTGGAGCTG
CTCCACGACGGGGCGCGTAGCAGCTGCATCCGTTGGACTGGCAACAGCCG
CGAGTTCCAGCTGTGCGACCCCAAAGAGGTGGCTCGGCTGTGGGGCGAGC
GCAAGAGAAAGCCGGGCATGAATTACGAGAAGCTGAGCCGGGGCCTTCGC
TACTACTATCGCCGCGACATCGTGCGCAAGAGCGGGGGGCGAAAGTACAC
GTACCGCTTCGGGGGCCGCGTGCCCAGCCTAGCCTATCCGGACTGTGCGG
GAGGCGGACGGGGAGCAGAGACACAATAAAAATTCCCGGTCAAACCTCAA
AAAAAAAAAAAAA

In certain embodiments, an exemplary VEGF polynucleotide sequence is and/or is comprised within the GENBANK® Accession No. AY047581 (SEQ ID NO: 13):

(SEQ ID NO: 13)
TCGGGCCTCCGAAACCATGAACTTTCTGCTGTCTTGGGTGCATTGGAGCC
TTGCCTTGCTGCTCTACCTCCACCATGCCAAGTGGTCCCAGGCTGCACCC
ATGGCAGAAGGAGGGGGGCAGAATCATCACGAAGTGGTGAAGTTCATGGA
TGTCTATCAGCGCAGCTACTGCCATCCAATCGAGACCCTGGTGGACATCT
TCCAGGAGTACCCTGATGAGATCGAGTACATCTTCAAGCCATCCTGTGTG
CCCCTGATGCGATGCGGGGGCTGCTGCAATGACGAGGGCCTGGAGTGTGT
GCCCACTGAGGAGTCCAACATCACCATGCAGATTATGCGGATCAAACCTC
ACCAAGGCCAGCACATAGGAGAGATGAGCTTCCTACAGCACAACAAATGT
GAATGCAGACCAAAGAAAGATAGAGCAAGACAAGAAAATCCCTGTGGGCC
TTGCTCAGAGCGGAGAAAGCATTTGTTTGTACAAGATCCGCAGACGTGTA
AATGTTCCTGCAAAAACACAGACTCGCGTTGCAAGGCGAGGCAGCTTGAG
TTAAACGAACGTACTTGCAGATGTGACAAGCCGAGGCGGTGAGCCGGGCA
GGAGGAAGGAGCCTCCCTCAGGGTTTCGGGAACCAGATCT

In particular embodiments, part or all of SEQ ID NOs: 12 and/or 13 is utilized in methods of the disclosure. In specific embodiments, a polynucleotide having a specific sequence identity with respect to SEQ ID NOs: 12 and/or 13 is utilized in methods of the disclosure. In specific cases, a functional fragment of SEQ ID NOs: 12 and/or 13 is employed, and the term “functional fragment” as used herein refers to a polynucleotide that encodes a polypeptide having the activity of being able to convert fibroblasts to endothelial cells or endothelial-like cells. In specific cases, the fragment has a length of at least about or no more than about 1375, 1350, 1325, 1300, 1275, 1250, 1225, 1200, 1175, 1150, 1125, 1100, 1075, 1050, 1025, 1000, 975, 950, 925, 900, 875, 850, 825, 800, 775, 750, 725, 700, 675, 650, 625, 600, 575, 550, 525, 500, 475, 450, 425, 400, 375, 350, 325, 300, 275, 250, 225, 200, 175, 150, 125, or 100 contiguous nucleotides of SEQ ID NOs: 12 and/or 13. In addition, the fragment may have sequence identity with the corresponding region in SEQ ID NOs: 12 and/or 13 of at least, or exactly, 100, 99, 98, 97, 96, 95, 94, 93, 92, 91, 90, 89, 88, 87, 86, 85, 80, 75, or 70% identity. A polynucleotide having certain sequence identity to SEQ ID NOs: 12 and/or 13 may be used, including at least, or exactly 100, 99, 98, 97, 96, 95, 94, 93, 92, 91, 90, 89, 88, 87, 86, 85, 80, 75, or 70% identity to SEQ ID NOs: 12 and/or 13.

In some embodiments, an ETV2 and/or VEGF polypeptide is delivered to an individual in need thereof, whether it be in the form of being on a vector, associated with a carrier, within a cell (including in a cell on a vector), and so forth. In specific embodiments, the ETV2 and/or VEGF polypeptide is a mammalian ETV2 and/or VEGF polypeptide, including human, mouse, rat, and so forth. In particular embodiments, one example of an ETV2 polypeptide sequence is and/or is comprised within the GENBANK® Accession No. NP_001287903 (SEQ ID NO: 14).

(SEQ ID NO: 14)
ACTAWDSWSGASQTLGPAPLGPGPIPAAGSEGAAGQNCVPVAGEATSWSR
AQAAGSNTSWDCSVGPDGDTYWGSGLGGEPRTDCTISWGGPAGPDCTTSW
NPGLHAGGTTSLKRYQSSALTVCSEPSPQSDRASLARCPKTNHRGPIQLW
QFLLELLHDGARSSCIRWTGNSREFQLCDPKEVARLWGERKRKPGMNYEK
LSRGLRYYYRRDIVRKSGGRKYTYRFGGRVPSLAYPDCAGGGRGAETQ

In particular embodiments, one example of a VEGF polypeptide sequence is and/or is comprised within the GENBANK® Accession No. AAK95847 (SEQ ID NO: 15).

(SEQ ID NO: 15)
MNFLLSWVHWSLALLLYLHHAKWSQAAPMAEGGGQNHHEVVKFMDVYQRS
YCHPIETLVDIFQEYPDEIEYIFKPSCVPLMRCGGCCNDEGLECVPTEES
NITMQIMRIKPHQGQHIGEMSFLQHNKCECRPKKDRARQENPCGPCSERR
KHLFVQDPQTCKCSCKNTDSRCKARQLELNERTCRCDKPRR

In particular embodiments, part or all of SEQ ID NOs: 14 and/or 15 is utilized in methods of the disclosure. In specific embodiments, a polypeptide having a specific sequence identity with respect to SEQ ID NOs: 14 and/or 15 is utilized in methods of the disclosure. In specific cases, a functional fragment of SEQ ID NOs: 14 and/or 15 is employed, and the term “functional fragment” as used herein refers to a polypeptide having the activity of being able to convert fibroblasts to endothelial cells or endothelial-like cells. In specific cases, the fragment has a length of at least about or no more than about 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, or 20 contiguous amino acids SEQ ID NOs: 14 and/or 15.

The agent that enhances angiogenesis may be referred to as an angiogenic factor. The agent may be provided to the individual prior to the individual receiving the p63-TID (or a functional fragment and/or a functional derivative thereof) and/or prior to the cardiac cell reprogramming factor and/or prior to the chromatin destabilizing agent. In certain embodiments, more than one agent is utilized.

The therapy of the present disclosure may precede or follow the other agent treatment by intervals ranging from minutes to hours to days to weeks or months. In embodiments where the other agent and the instant therapy are applied separately to the individual, one would generally ensure that a significant period of time did not expire between the time of each delivery, such that the therapy of the disclosure and the additional therapy would still be able to exert an advantageously combined effect on the individual. In such instances, it is contemplated that one may contact the individual with both modalities simultaneously or within minutes of each other or within about 1-12, 6-12, or 12-24 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In specific embodiments, the therapy of the present disclosure and the additional therapy are provided at the same time or at different times. The separate entities may be within the same compositions or they may be comprised in separate compositions. In cases wherein the therapy of the present disclosure and the second therapy are provided at different times, they may be separated by any suitable range in times, such as minutes, hours, days, or weeks. In embodiments wherein they are provided separately, the order of delivery of two (or more) therapies may be of any suitable order, including delivery of p63-TID (or a functional fragment and/or a functional derivative thereof) with Hand2 and/or myocardin prior to or subsequent to another therapy.

Examples of other treatments to be employed with the therapy of the disclosure includes one or more of the following: ACE Inhibitors, Aldosterone Inhibitor, Angiotensin II Receptor Blocker (ARBs); Beta-Blockers, Calcium Channel Blockers, Cholesterol-Lowering Drugs, Digoxin, Diuretics, Inotropic Therapy, Potassium or Magnesium, Vasodilators, anticoagulant medication, aspirin, surgery, VAD implantation, VAT, coronary bypass, percutaneous coronary intervention (PCI) or a combination thereof.

### X. Kits of the Disclosure

Any of the compositions described herein may be comprised in a kit. In a non-limiting example, p63-TID (or a functional fragment and/or a functional derivative thereof), one or more cardiac cell reprogramming factors, and/or one or more chromatin destabilizing agents or other polynucleotide or primers for amplification of same may be comprised in a kit. In specific embodiments, the kit comprises p63-TID (or a functional fragment and/or a functional derivative thereof) with or without Hand2 and/or myocardin polypeptides or peptides. One or more reagents to generate p63-TID (or a functional fragment and/or a functional derivative thereof) or one or more of the other mentioned factors may be included in the kit, such as specific primers to amplify the desired sequence. In such cases, the kit may or may not comprise standard reagents for such a method, such as nucleotides, buffers, etc. The kit may additionally comprise additional agents for therapy of a cardiac medical condition.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the p63-TID (or a functional fragment and/or a functional derivative thereof) and one or more compositions in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly considered. The p63-TID (or a functional fragment and/or a functional derivative thereof) compositions may also may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

The kits of the present disclosure will also typically include a means for containing the vials in close confinement for commercial sale, such as, e.g., injection and/or blow-molded plastic containers into which the desired vials are retained.

In particular embodiments, the kit comprises reagents and/or tools for determining that an individual has a cardiac medical condition. In some embodiments, the kit comprises one or more additional therapies for a cardiac-related medical condition, such as one or more of ACE Inhibitor, aldosterone inhibitor, angiotensin II receptor blocker (ARBs); beta-blocker, calcium channel blocker, cholesterol-lowering drug, digoxin, diuretics, inotropic therapy, potassium, magnesium, vasodilator, anticoagulant medication, aspirin, TGF-beta inhibitor, and a combination thereof. In specific embodiments, an individual receives angiogenic therapy before, during, or after the therapy of the present disclosure. Examples of angiogenic therapies include fibroblast growth factor (FGF); vascular endothelial growth factor (VEGF); Ets variant 2 (ETV2); angiopoietins, Ang1 and Ang2; matrix metalloproteinase (MMP); Delta-like ligand 4 (DII4); or peptides thereof; or combinations thereof.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Methods

Unless otherwise noted, the experiments and procedures described herein were conducted as follows.

### Tissue Collection and Isolation of Cardiac Fibroblasts

Neonatal and adult cardiac fibroblasts were harvested using standard cell isolation techniques from 0-3 day-old to 6-8 week-old rats, respectively (Harlan Sprague Dawley Inc, Indianapolis, IN) (see e.g., citations 9, 10, and 27, each of which are incorporated herein by reference for the purposes described herein). All animal experiments were approved by Institutional Animal Care and Use Committee (IACUC) at Baylor College of Medicine and all methods were carried out in accordance with the NIH guidelines (Guide for the care and use of laboratory animals) and under protocol AN-6223. These studies were conducted and are reported in compliance with relevant elements of ARRIVE guidelines.

Adult human cardiac fibroblasts were isolated using standard isolation techniques from ventricular myocardial tissue obtained from explants of heart failure patients undergoing mechanical assist device placement or cardiac transplantation at Baylor St. Luke's Medical Center (see e.g., citations 9, and 10, each of which are incorporated herein by reference for the purposes described herein). A written informed consent was obtained from all the subjects and/or their legal guardian(s) prior to obtaining the tissue. All experimental methods were carried out in accordance with relevant guidelines and regulations under a protocol approved by the Baylor College of Medicine Institutional Review Board (IRB H-33421). Briefly, explanted tissues were minced and then cultured in DMEM, 10% fetal bovine serum (FBS) and 1% penicillin/streptomycin. Fibroblasts were thereby allowed to migrate out from these explants over a period of 2 weeks, after which they were passaged three times in M106 medium (M106500; Thermo Fisher Scientific), 10% FBS, and LSGS kit supplements (S-003-K; Thermo Fisher Scientific).

### Cell Reprogramming

Lentivirus vectors each encoding Gata4, Mef2, or Tbx5 (GMT), Hand2/Myocardin (H/M), non-targeting (NT) shRNA, p63 short hairpin RNA (Origene, Rockville, MD), p63-transactivation inhibitory domain (Vectorbuilder, Chicago, IL) tagged with green fluorescent protein (GFP) or GFP control vectors were prepared from relevant plasmids by the Baylor College Of Medicine Gene Vector Core, as previously described (see e.g., citations 9, 10, 27, and 28, each of which are incorporated herein by reference for the purposes described herein).

Rat and human cardiac fibroblasts isolated as described above were seeded onto 6 cm or 10 cm culture dishes for fluorescence-activated cell sorting [FACS] analyses, onto 6-well plates for quantitative reverse transcription polymerase chain reaction [qRT-PCR] analysis or onto 24-well dishes pre-coated with Surecoat (SC-9035; Cellutron Life Technologies) for immunocytochemistry analyses. Twenty four hours after the cells were 70% to 80% confluent, lentiviral vectors at a multiplicity of infection (MOI) of 20 (unless otherwise indicated) were added to cell culture plates in a mixture with polybrene at a final concentration of 5 μg/μL. Two days after cell culture treatment with relevant reprogramming factors, the initial transfer medium (DMEM/199 [4:1], 10% FBS, and 1% penicillin/streptomycin) was replaced with induction medium (iCM media), as previously described. This media was replaced with fresh induction media every two days until cells were harvested (see e.g., citations 9, and 10, each of which are incorporated herein by reference for the purposes described herein).

For cell contractility co-culture studies, cardiomyocytes were isolated from 0 to 3 day old rat pups under protocol AN-6223, as previously described (see e.g., citations 28-30, each of which are incorporated herein by reference for the purposes described herein). Human cardiac fibroblasts were treated with GFP-labeled reprogramming factors (e.g., GMT, shp63+ H/M, p63-TID+ H/M) and one week after treatment as described above, cells were harvested and re-plated onto neonatal rat cardiomyocytes at a ratio of 1:10 and cultured in DMEM/M-199/10% FBS medium (see e.g., citation 31, which is incorporated herein by reference for the purposes described herein).

### Flow Cytometry

Fluorescence-activated cell sorting (FACS) was performed as previously described (see e.g., citations 8-10, 27, and 28, each of which are incorporated herein by reference for the purposes described herein). Briefly, cells adherent to culture dishes were first washed with DPBS and trypsinized with 0.25% trypsin/EDTA. Cells were then fixed with fixation buffer (BD Biosciences), washed with Perm/Wash buffer (BD Biosciences) and then incubated with mouse monoclonal anti-cardiac troponin T (cTnT) antibody (ab8295; Abcam) in Perm/Wash buffer. These cells were then incubated with donkey anti-mouse Alexa Fluor 647 (ab150107; INVITROGEN™), washed 3x with Perm/Wash buffer again, and further analyzed for cTnT expression using a LSR Fortessa cell sorter (BD Biosciences) with FlowJo software (FlowJo, LLC, Ashland, Ore) and Diva software (version 6.0).

### Immunocytochemistry

Immunofluorescence (IF) staining was performed using cells fixed in 4% paraformaldehyde and permeabilized with 0.5% Triton-X solution, as previously described (see e.g., citations 8-10, and 28, each of which are incorporated herein by reference for the purposes described herein). After these cells were blocked with 10% goat serum, they were incubated with primary antibodies against cTnT (1:300 dilution; Thermo Fisher Scientific), or α-actinin (1:300 dilution; Sigma-Aldrich) followed by incubation with appropriate Alexa fluorogenic secondary antibodies (INVITROGEN™). 4′,6-diamidino-2-phenylindole (DAPI; INVITROGEN™) was used to stain nuclei. For quantification of cTnT and α-actinin positive cells, the ratio of cells expressing relevant IF markers versus total cells marked by DAPI was calculated in five random images selected by an investigator blinded to treatment group.

qRT-PCR

Quantitative real-time polymerase chain reaction (qRT-PCR) analysis was performed by first extracting total RNA using the TRIzol method (INVITROGEN™), as previously described (see e.g., 8-10,28,32). Relative quantification of RNA was performed using SYBR green detection of PCR products in real time with the ABI ViiA 7 (Applied Biosystems Inc). Primers for qRT-PCR used in this study are listed in Table 1 (Cardiac muscle isoform of troponin T (cTnT); Ryanodine receptor (RyR); Phospholamban (PIn); Actin Alpha Cardiac Muscle 1 (Actc1); Collagen type 1 alpha 1 chain (Collal); Periostin (Postn); Glyceraldehyde-3-phosphate dehydrogenase (Gapdh); Delta N isoform of tumor protein p63 (ΔNp63); Transactivation (TA) isoform of tumor protein p63 (TAp63); Myosin heavy chain 6 (Myh6); Gap junction alpha-i (Gja1)). mRNA levels were normalized by comparative ΔΔCT method with comparison to glyceraldehyde 3-phosphate dehydrogenase (GAPDH).

TABLE 1
qRT-PCR primers

| Organism | Gene | Forward Primer (SEQ ID NO) | Reverse Primer (SEQ ID NO) |
|---|---|---|---|
| Rat | cTnT | AGGCTCACTTCGAGAACAGG | ATTGCGAATACGCTGCTGT |
| | | (SEQ ID NO: 16) | (SEQ ID NO: 17) |
| Rat | Ryr | ACATCATGTTTTACCGCCTGAG | TTTGTGGTTATTGAACTCTGGCT |
| | | (SEQ ID NO: 18) | (SEQ ID NO: 19) |
| Rat | Pln | GTGACGATCACAGAAGCCAAGG | TGACAGCAGGCAGCCAAACG |
| | | (SEQ ID NO: 20) | (SEQ ID NO: 21) |
| Rat | Actc1 | GATTATTGCTCCCCCTGAGCG | GTGTAAGGTAGCCGCCTCAGAA |
| | | (SEQ ID NO: 22) | (SEQ ID NO: 23) |
| Rat | Colla1 | GCGAAGGCAACAGTCGATTC | CCCAAGTTCCGGTGTGACTC |
| | | (SEQ ID NO: 24) | (SEQ ID NO: 25) |
| Rat | Postn | GGCTGAAGACTGCCTTGAATGAC | CGTGGCAGCACCTTCAAAGA |
| | | (SEQ ID NO: 26) | (SEQ ID NO: 27) |
| Rat | Gapdh | GGCACAGTCAAGGCTGAGAATG | ATGGTGGTGAAGACGCCAGTA |
| | | (SEQ ID NO: 28) | (SEQ ID NO: 29) |
| Rat | ΔNp63 | GGAAAACAATGCCCAGACTC | GTGGAATACGTCCAGGTGGC |
| | | (SEQ ID NO: 30) | (SEQ ID NO: 31) |
| Rat | TAp63 | AAGATGGTGCGACAAACAAG | AGAGAGCATCGAAGGTGGAG |
| | | (SEQ ID NO: 32) | (SEQ ID NO: 33) |
| Human | cTnT | AGACGCCTCCAGGATCTGT | TCTTCAACAGCTGCTTCTTCC |
| | | (SEQ ID NO: 34) | (SEQ ID NO: 35) |
| Human | Myh6 | GCTGGTCACCAACAATCCCTA | CGTCAAAGGCACTATCGGTGG |
| | | (SEQ ID NO: 36) | (SEQ ID NO: 37) |
| Human | Gja1 | GCCAAAGACTGTGGGTCTCA | GAAGGTCGCTGGTCCACAAT |
| | | (SEQ ID NO: 38) | (SEQ ID NO: 39) |
| Human | Colla1 | CATGTTCAGCTTTGTGGACCT | GCAGCTGACTTCAGGGATGT |
| | | (SEQ ID NO: 40) | (SEQ ID NO: 41) |
| Human | Postn | CTCATAGTCGTATCAGGGGTCG | ACACAGTCGTTTTCTGTCCAC |
| | | (SEQ ID NO: 42) | (SEQ ID NO: 43) |
| Human | Gapdh | TCACCACCATGGAGAAGGC | GCTAAGCAGTTGGTGGTGCA |
| | | (SEQ ID NO: 44) | (SEQ ID NO: 45) |
| Human | ΔNp63 | GAAAACAATGCCCAGACTCAA | TGCGCGTGGTCTGTGTTA |
| | | (SEQ ID NO: 46) | SEQ ID NO: 47) |
| Human | TAp63 | TGTATCCGCATGCAGGACT | CTGTGTTATAGGGACTGGTGGAC |
| | | (SEQ ID NO: 48) | (SEQ ID NO: 49) |

### Co-Immunoprecipitation (Co-IP) and Western Analyses

For Western analyses and Co-IP, 293T cells were transfected with plasmid vectors pcDNA3.1, ΔNp63 α-FLAG (p63-FLAG; #26979, Addgene), HDAC1-GFP (#11054, Addgene) or p63-TID-HA (GenScript®) in LIPOFECTAMINE™ 3000 Transfection Reagent (L3000008, Thermo Fisher Scientific). Cell lysates were collected and homogenized in cell lysis buffer. Protein was quantified using Pierce BCA protein assay kit (23227; Thermo Fisher Scientific) and Co-IP was performed with quantified protein using Immunoprecipitation Kit (10007D; INVITROGEN™) following manufacturer's protocol. As a final step, samples were loaded onto SDS-PAGE and after separation, the protein bands were transferred to nitrocellulose membrane (I1B301001; INVITROGEN™).

Immune detection was performed with the following primary antibodies: FLAG tag (F1804-200UG; Sigma-Aldrich), HDAC1 (sc-7872; Santa Cruz Biotechnology, Inc), β-Actin (sc-47778; Sigma-Aldrich), HA tag (sc-57592; Santa Cruz Biotechnology, Inc,), or TP63 (GTX 102425; GeneTex), followed by treatment with appropriate HRP-conjugated secondary antibodies (Millipore, Billerica, MA). Membranes were then washed with 1× Trisbuffered saline with Tween 20 and visualized by chemiluminescence detection (WBLUF0500; Millipore Sigma).

### Measurements of Contractility and Calcium Transient

Cell contractility (cell shortening) and calcium transients in co-culture studies were measured at room temperature (22-23° C.). To perform these studies, cells were placed in plexiglass chamber which was positioned on the stage of an inverted epifluorescence microscope (Nikon Diaphot 2000) and perfused with 1.8 mmol/L Ca²⁺-Tyrode's solution containing (in mmol/L): NaCl 140, KCl 5.4, MgCl₂ 1, CaCl₂) 1.8, HEPES 5, and glucose 10, pH 7.4. Cells that had been previously treated with reprogramming factors were identified by GFP fluorescence.

Field-stimulation was provided by a Grass S5 stimulator using platinum electrodes placed alongside a cell culture bath containing 1.8 mM Ca²⁺, with bipolar pulses delivered at voltages 50% above myocyte stimulation thresholds. Contractions of iCMs from random fields were videotaped and digitized on a computer. For Ca2⁺ signal measurements, cells were loaded with 3 μmol/L of Fura-2/AM (Life Technologies) and alternately excited at 340 and 380 nm at 0.5 Hz by use of a Delta Scan dual-beam spectrophotofluorometer (Photon Technology International, Edison, NJ). Ca2⁺ transients were expressed as the 340/380-nm ratios of the resulting 510-nm emissions. Data were analyzed using Felix software (Photon Technology International) (see e.g., citations 8, 9, and 28-30, each of which are incorporated herein by reference for the purposes described herein).

### Statistical Analyses

At least three independent biological replicates each measured in technical triplicates were performed for all studies. All data are expressed as the mean±standard error (SEM). Statistical analysis was performed using SAS, version 9.4. Student's t-test was used to determine significance of differences between two groups. One-way ANOVA was used to determine the significance of differences when more than 2 groups were compared.

### Example 1

### A Shortened P63-Protein Domain Enhanced Human Cardiac Reprogramming

Direct reprogramming represents a promising new strategy for treating heart failure by causing the in situ transdifferentiation of cardiac fibroblasts into functional cardiomyocyte-like cells (iCMs). Notably, a combination of three cardiac transcription factors Gata4, Mef2c and Tbx5 can convert the fibroblasts to iCMs. Recent findings suggest, however, that human cells are resistant to reprogramming compared to rodent cells, likely because of epigenetic restraints on reprogramming gene activation. The present disclosure concerns enhancement of the cardiac reprogramming efficiency and maturation of iCMs by the modulation (e.g., inhibition; e.g., silencing of the epigenetic effects) of the epigenetic regulator gene p63, which has been shown to enhance pluripotent stem cell differentiation. The data shows that p63-Transactivation Inhibitory domain (TID; p63-TID) exerts reprogramming benefits.

The family of histone deacetylases (HDACs) remove acetylation groups and result in the formation of a condensed and more silenced chromatin transcriptionally on cardiac transdifferentiation genes. In fact, there is evidence that HDAC inhibitors enhance cardiac reprogramming. It was considered in the present disclosure if p63-HDAC1 complex could play a primary role in influencing the gene transcription of cardiac transcription factors.

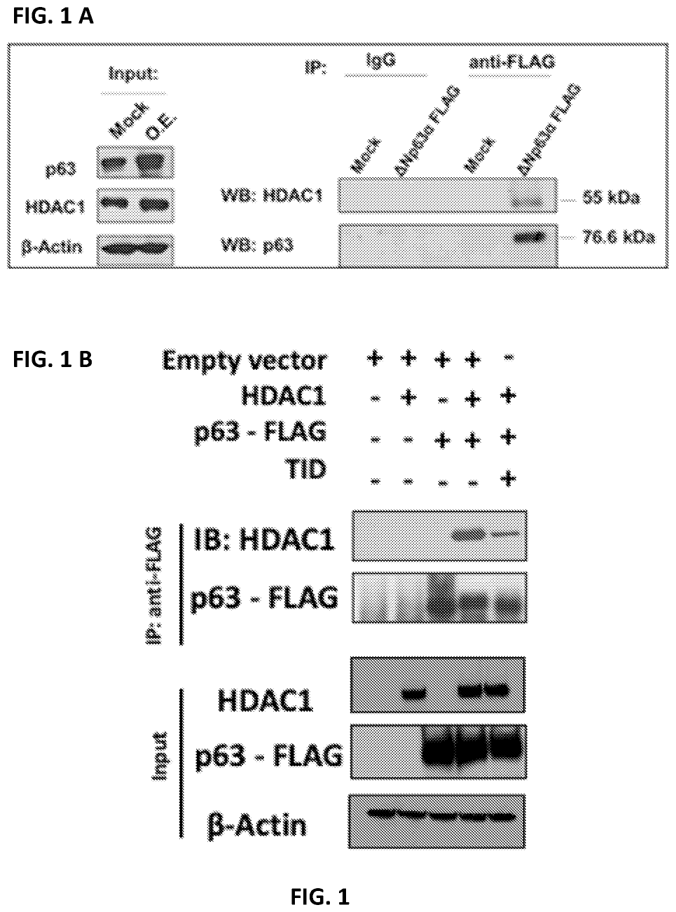

As shown in FIG. 1 A, the inventors validated by a co-immunoprecipitation (co-IP) assay the p63-HDAC1 interaction. Further, it was considered if overexpressing p63-TID acts to compete the protein-protein interactions between p63 & HDAC1 such that consequently their epigenetic interactions could specifically be targeted by using p63-TID. As seen in FIG. 1 B, it was demonstrated by co-IP pulldown assay that p63-TID fragment overexpression reduces the binding between ΔNP63α and HDAC1 by competitively binding to HDAC1.

An exemplary p63-TID peptide (e.g., SEQ ID NO: 1) nucleotide coding sequence comprising an optional tag (e.g., HA tag) (SEQ ID NO: 2) and nucleotide vector comprising the same were obtained and are displayed in FIGS. 4 A-B.

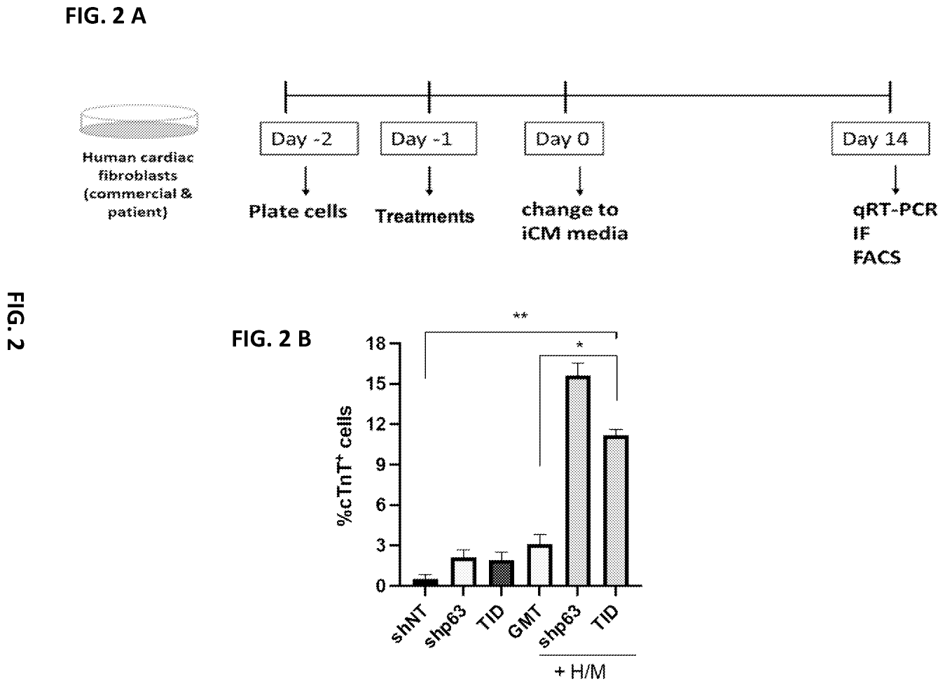

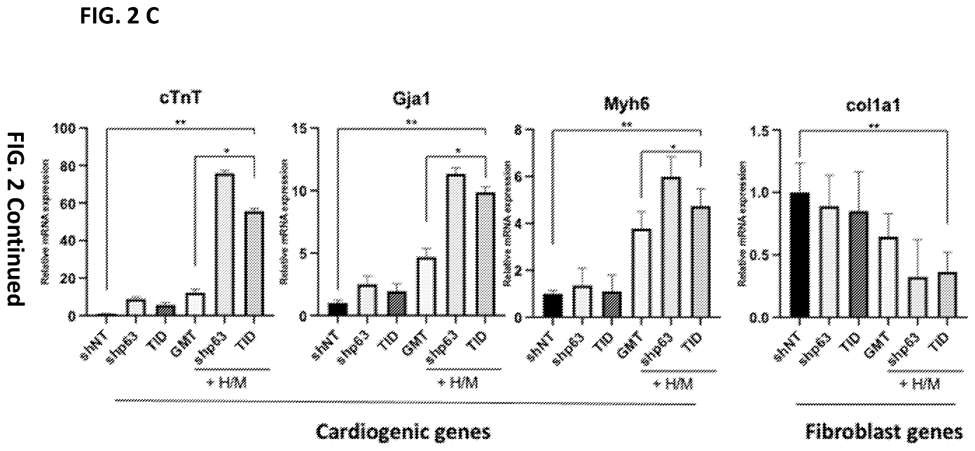

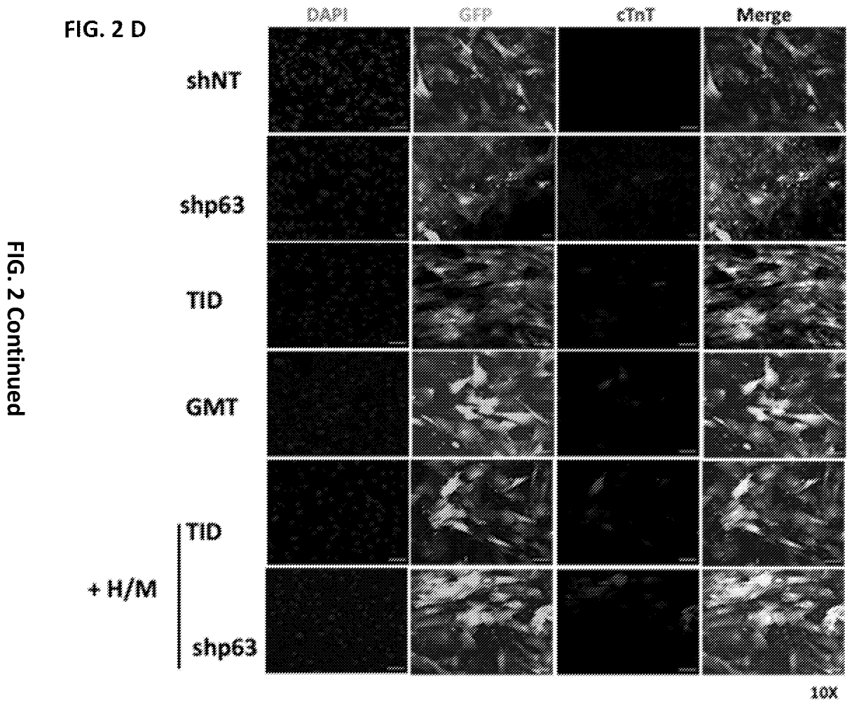

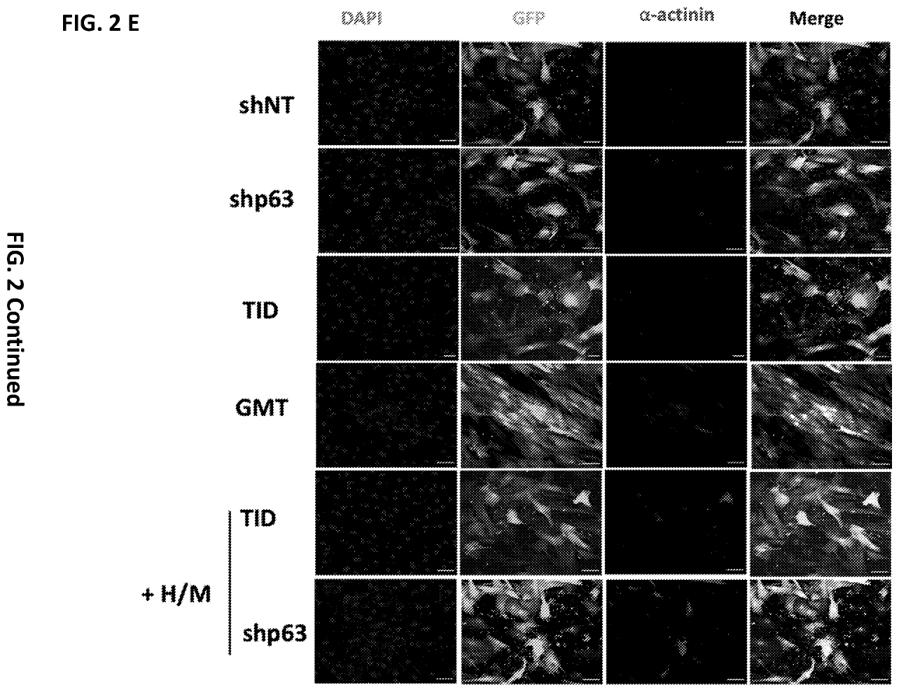

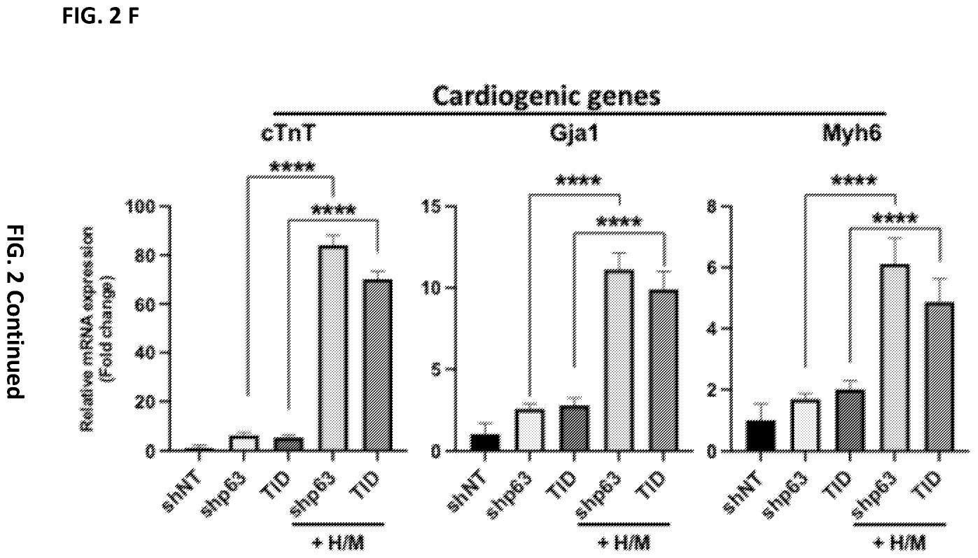

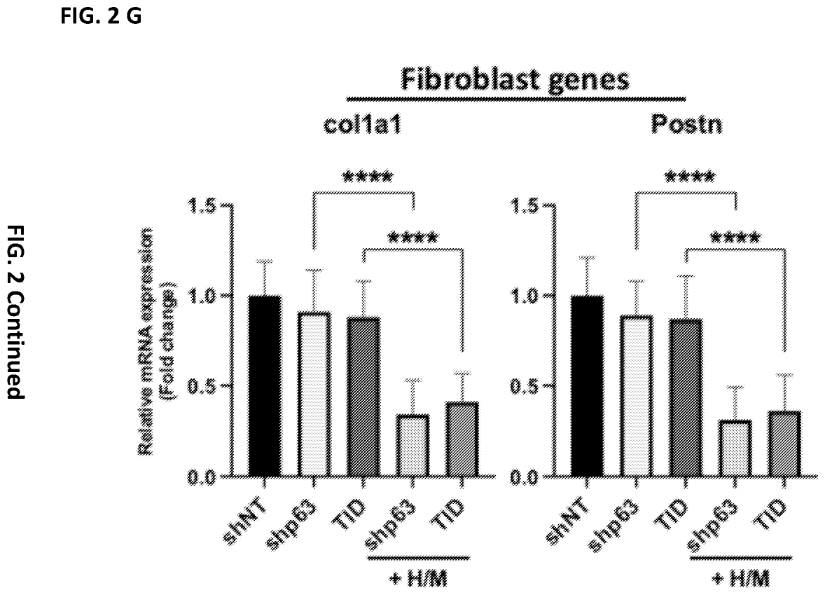

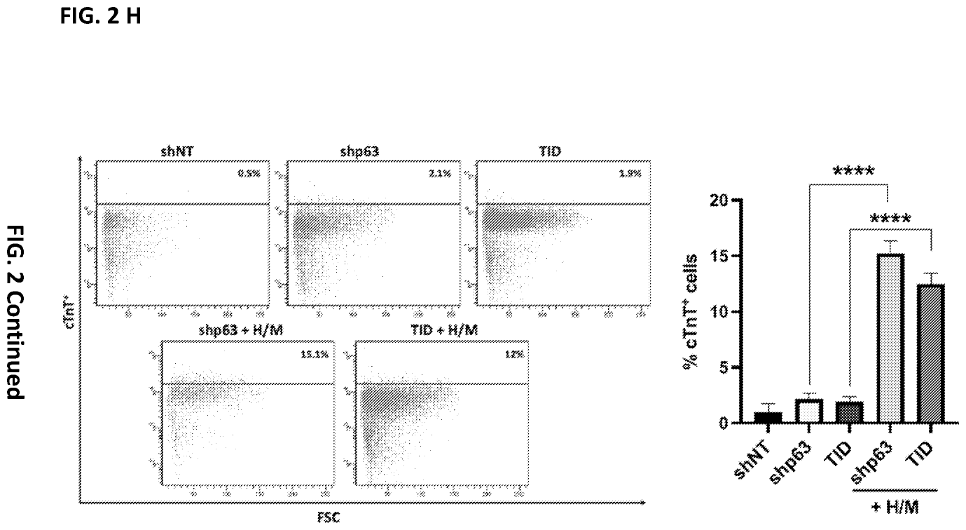

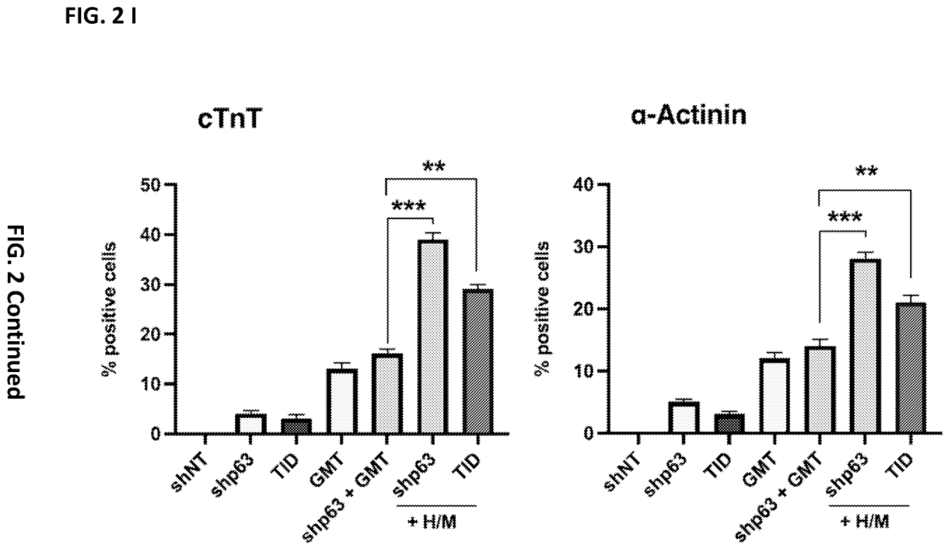

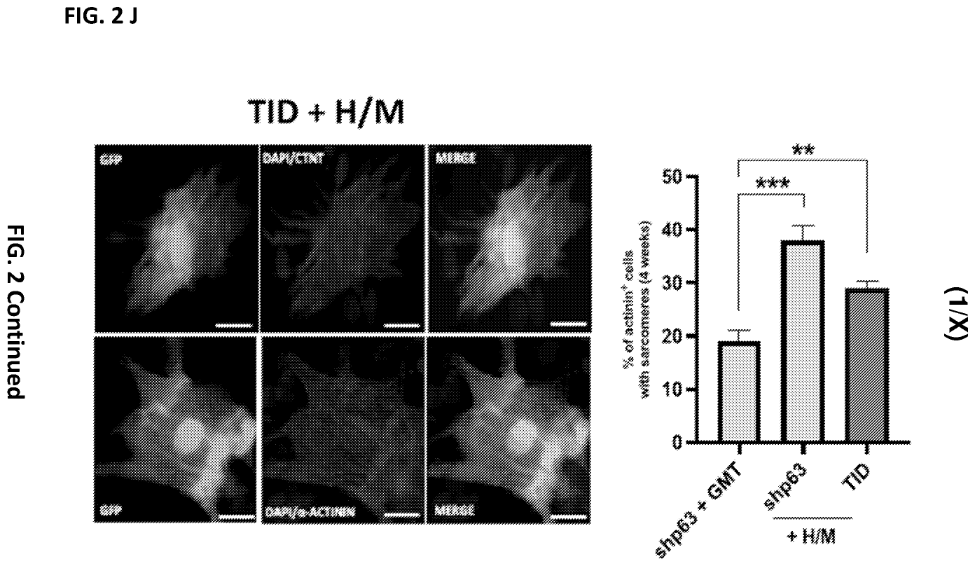

The inventors then substantiated the effect of p63-TID on cardiac reprogramming by treating human cardiac fibroblasts with lentivirus encoding p63-TID±Hand2/Myocardin. After 14 days of culturing, cells were assessed for cardiomyocyte-specific feature changes using qRT-PCR, flow cytometry, and immunofluorescence assays. Lentiviral-mediated p63-TID overexpression in combination with Hand2/Myocardin administration to human cardiac fibroblasts upregulated the same panel of cardiac genes as seen with p63 shRNA+Hand2/Myocardin ((+H/M) FIGS. 2 A-J), and it was observed that p63-TID overexpression similarly increased the percentage of human cardiac fibroblasts expressing cTnT and a-Actinin (FIGS. 2 A-J). Cells treated with p63-TID+ H/M displayed increased expression of a panel of cardiomyocyte marker genes (cTnT, Gja1, Myh6) similar to that induced by shp63+ H/M (FIG. 2 I) and reduced expression of fibroblast marker genes (colla1, Postn) comparable to that achieved with shp63+ H/M treatment (FIG. 2 G). Interestingly, p63-TID overexpression alone upregulated expression of a panel of genes that favor cardiac differentiation (e.g., CTnT, Gja1, α-Actinin, and Myh6), and likewise downregulated genes associated with fibroblast signature (e.g., colla1, and Postn).

Exemplary p63-TID (e.g., SEQ ID NO: 1) was also administered to rat cardiac fibroblasts and yielded better iCM reprogramming outcomes when compared to controls. As displayed in FIG. 3, mRNA expression levels of indicated cardiac markers (e.g., cTnT, RyR, Pln, and Actc1) and fibroblast marker gene (e.g., colla1) mRNA expression two weeks after reprogramming factors administration were determined by qRT-PCR. Significant increases in cardiogenic gene expression levels were observed with p63-TID+ H/M administration, while decreases in fibroblast marker colla1 were also observed.

In addition, FACS analysis similarly demonstrated that the percentage of human cardiac fibroblasts expressing cTnT was similarly increased after treatment with p63-TID+ H/M compared to shp63+ H/M treatment (12.5%±0.9% and 15.2%±1.1%, FIG. 2 H). Immunofluorescence studies also demonstrated a similar threefold increase in the number of cells expressing the cardiomyocyte markers cTnT and α-actinin after treatment with p63-TID+ H/M or shp63+ H/M versus cells treated with shp63+ GMT (p<0.001; FIGS. 2 D, E, and I). Four weeks after reprogramming factor treatment, threefold more cells treated with p63-TID+ H/M exhibited α-sarcomeric actinin+expression and p63-TID+ H/M treated cells exhibited advanced sarcomere organization compared to cells treated with shp63 and GMT (FIG. 2 J).

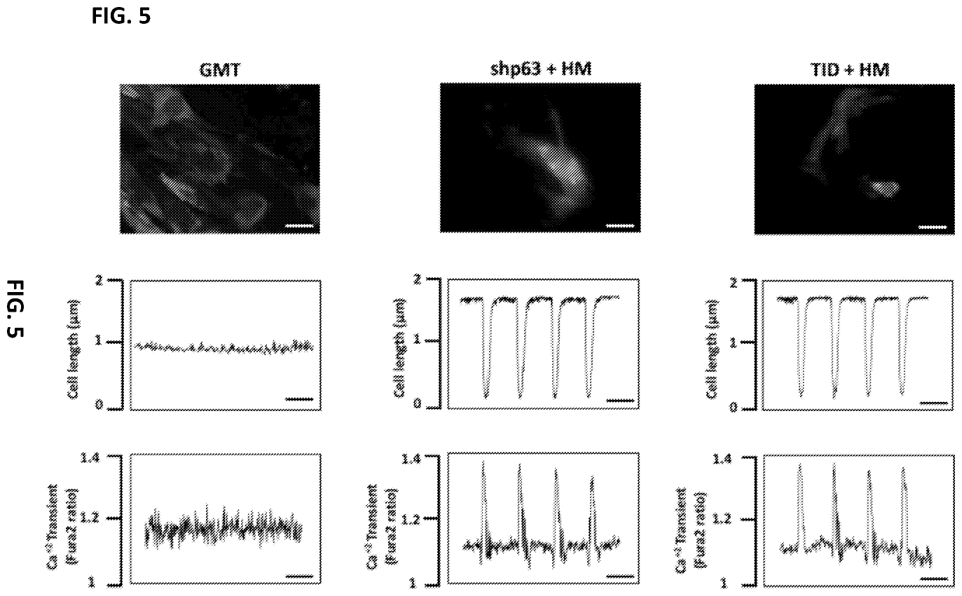

The inventors then determined that p63 silencing induced iCM contractility. Although human cardiac fibroblasts treated with shp63+ H/M or p63-TID+ H/M were not observed to contract independently, approximately ˜ 5% of human cardiac fibroblasts treated with shp63+H/M or TID+ H/M, as verified by their GFP expression, contracted synchronously with surrounding neonatal rat cardiomyocytes after 4 weeks in co-culture (FIG. 5). In comparison, human cardiac fibroblasts treated with GMT with or without shp63 failed to contract in co-culture experiments (see e.g., Pinnamaneni et al., p63 silencing induces epigenetic modulation to enhance human cardiac fibroblast to cardiomyocyte-like differentiation. Scientific Reports (2022)12:11416; which is incorporated herein by reference in its entirety for the purposes described herein, Supplemental Videos S1, S2, and/or S3). Cells treated with shp63+ H/M or p63-TID+ H/M also demonstrated calcium transients upon electrical stimulation that was synchronous with their contractile function, whereas calcium transients were not observed after stimulation of cells treated with GMT with or without shp63 (FIG. 5, middle and bottom rows; and Pinnamaneni et al., 2022, Supplemental Video S1, S2, S3, which are incorporated herein by reference in their entirety for the purposes described herein).

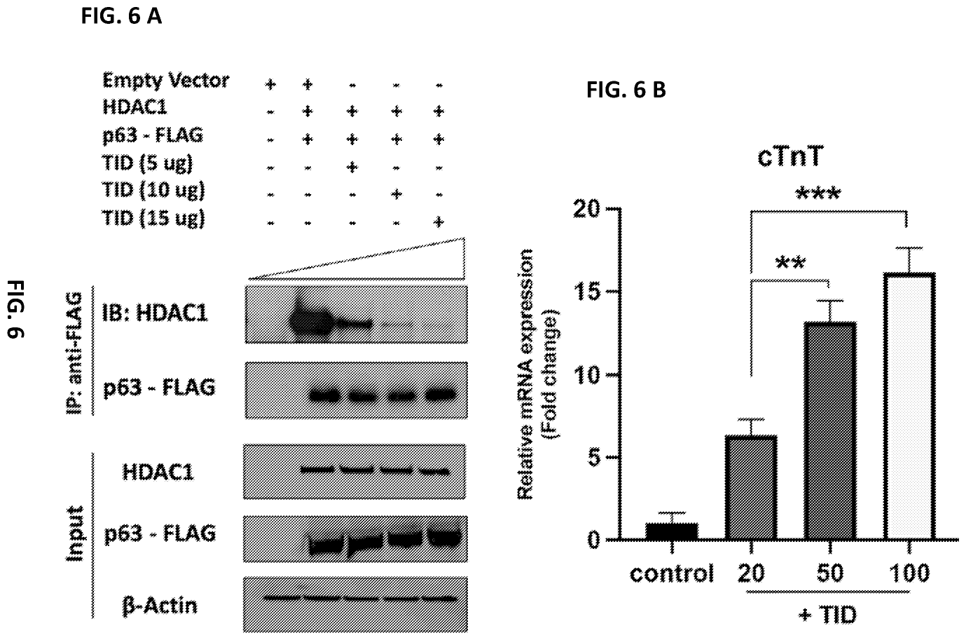

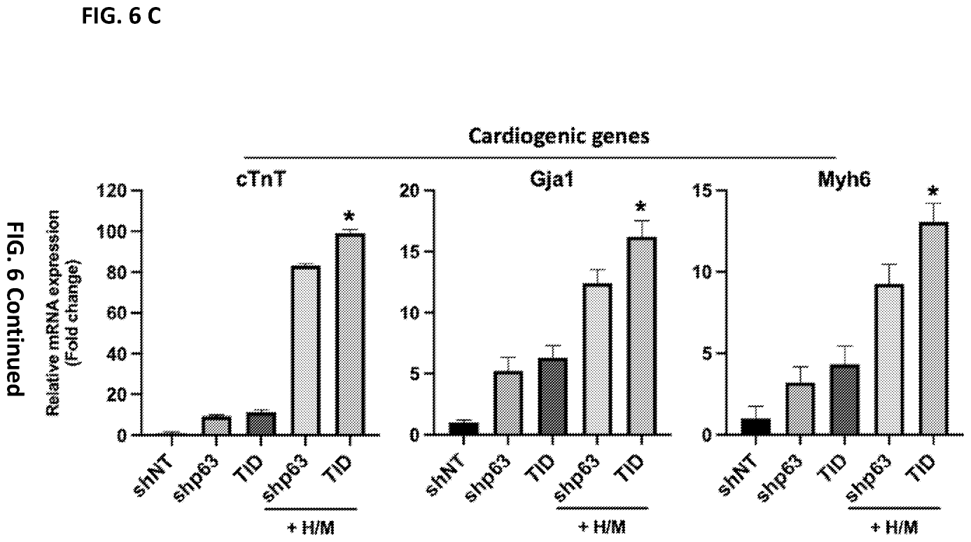

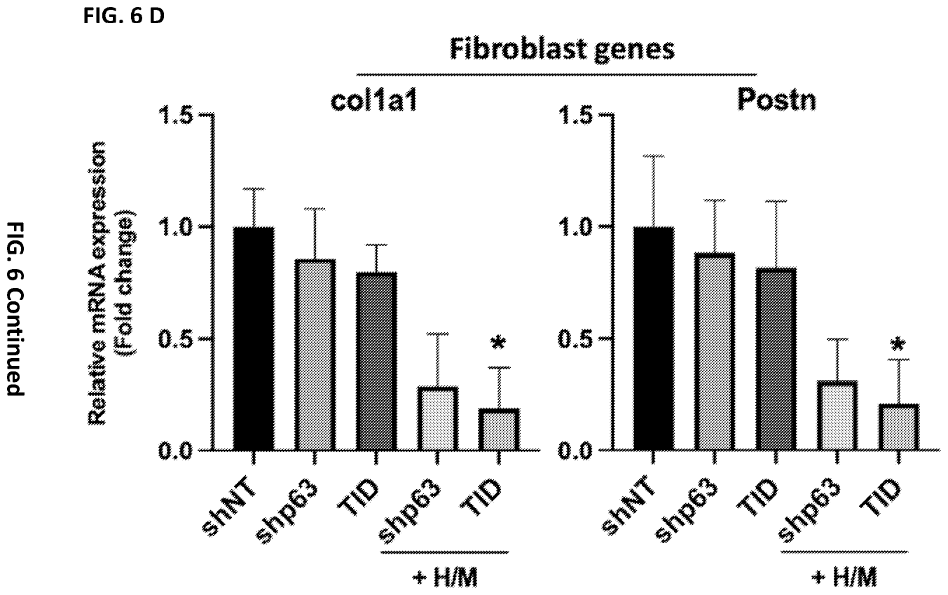

The inventors then determined p63-TID dose responses, and identified enhanced p63-TID potency. when compared to shp63. in enhancing human cardio-differentiation. To determine whether p63-TID was more potent than shp63 in enhancing cardio-differentiation, the inventors utilized Co-IP analysis to generate a dose-response analysis of p63 binding to HDAC1 as a function of p63-TID overexpression (FIG. 6 A). qRT-PCR analysis of human cardiac fibroblasts treated with p63-TID at an MOI of 20, 50 or 100 MOI demonstrated increased cTnT expression in a dose-dependent fashion, with an MOI of 50 providing the highest cTnT expression without cell toxicity (p<0.001; FIG. 6 B). The inventors were accordingly able to use qRT-PCR of human cardiac fibroblasts treated at an MOI of 50 to demonstrate significantly greater changes in cardiogenic and fibrogenic gene expression after p63-TID+ H/M versus shp63+ H/M treatment (p<0.05, FIG. 6 C-D).

As described herein and in Pinnamaneni et al., 2022, the inventors showed that rodent as well as human cardiac fibroblasts can be converted into contractile iCMs through a reprogramming strategy mediated by the silencing of the epigenetic effects of p63. Specifically, the inventors demonstrated that shp63 in combination with the cardio-differentiation factors Hand2 and Myocardin (H/M) led to enhanced neonatal, adult rat and adult human cardiac fibroblast differentiation compared to their treatment with a standard reprogramming cocktail (i.e., Gata4, Mef2c and Tbx5 [GMT]) alone (see e.g., citations 5, 10, and 34, each of which are incorporated herein by reference for the purposes described herein). In comparison, neither shp63 nor H/M alone exerted significant reprogramming effects.

The focus on p63 described in these studies stemmed from observations that the p53 family of epigenetic regulator proteins play an important role in impeding induced pluripotent stem cell (iPSC) reprogramming (see e.g., citations 18-20, 23, 35, and 36, each of which are incorporated herein by reference for the purposes described herein). The inventors hypothesized that silencing of p63, which appears to play a role similar to p53 in repressing iPSC reprogramming without its oncogenic effects, might be an ideal reprogramming agent for the enhancement of cardiac cell transdifferentiation and iCM generation (see e.g., citations 24, 37-43, each of which are incorporated herein by reference for the purposes described herein). The results described herein and in Pinnamaneni et al., 2022, confirmed the inventors hypothesis and specifically identified p63 interactions with the epigenetic repressor HDAC1 as a potential mechanism of action underlying this effect. The finding that p63 interacts with HDAC1 to initiate epigenetic re-patterning and modulate cardio-differentiation gene promoters confirmed the role of epigenetic modulation as an important regulator of human cell cardiac reprogramming (see e.g., citations 44, and 45, each of which are incorporated herein by reference for the purposes described herein).

The C-terminus of both of the two major isoforms of p63 (TAp63, ΔNp63) contains a transactivation inhibitory domain (TID) that has been reported to play an important role in gene regulation via its interactions with HDAC1 (see e.g., citation 26, which is incorporated herein by reference for the purposes described herein). The inventors consequently hypothesized that overexpression of TID could substitute for the use of shRNA to inhibit the epigenetic effects of p63 and associated inhibition of cell reprogramming. The results provided herein demonstrated that p63-TID could be used in this manner, and enhanced cardiogenic reprogramming gene activation. While not being limited by theory, the potency of the p63 silencing strategy in inducing contractile iCMs compared to the use of a standard reprogramming cocktail could be related to its observed effects in influencing the regulation of a diverse panel of relevant cardiogenic and fibrogenic genes. In comparison, standard reprogramming cocktails have required the administration of each of these constituent reprogramming factors in order to achieve efficacy (see e.g., citations 11, 31, 34, and 46-48, each of which are incorporated herein by reference for the purposes described herein). In this context, while not being limited by theory, the addition of H/M as a supplement to p63 silencing likely relates to status of H/M as the “missing element” complementing key cardio-differentiation factors such as GMT that are otherwise upregulated by the described p63 silencing strategy. It is likewise interesting that p63 silencing lead to the downregulation of fibrogenic genes known to impede cardio-differentiation, which others have addressed by addition of additional potentially undesirable anti-fibrogenic factors to reprogramming cocktails (see e.g., citations 9, 10, and 46-48, each of which are incorporated herein by reference for the purposes described herein).

In sum, the results described and demonstrated herein showed that overexpression of the p63-Transactivation inhibitory domain (TID), the p63 motif responsible for binding to the epigenetic regulator histone deacetylase 1 (HDAC1), was a potent replacement for shp63 in enhancing human cardio-differentiation. Furthermore, the results presented herein suggest that p63 acts as an epigenetic barrier to human cardiac reprogramming, and that p63-TID offers a new potential strategy to target epigenetic regulation of cardiogenic gene activation as a means to enhance human cardiac reprogramming and/or treat and/or prevent diseases described herein (e.g., but not limited to, cardiac associated indications).

### Example 2

### P63-TID with HAND2/Myocardin and ETV2 or VEGF Enhanced Human Cardiac Reprogramming

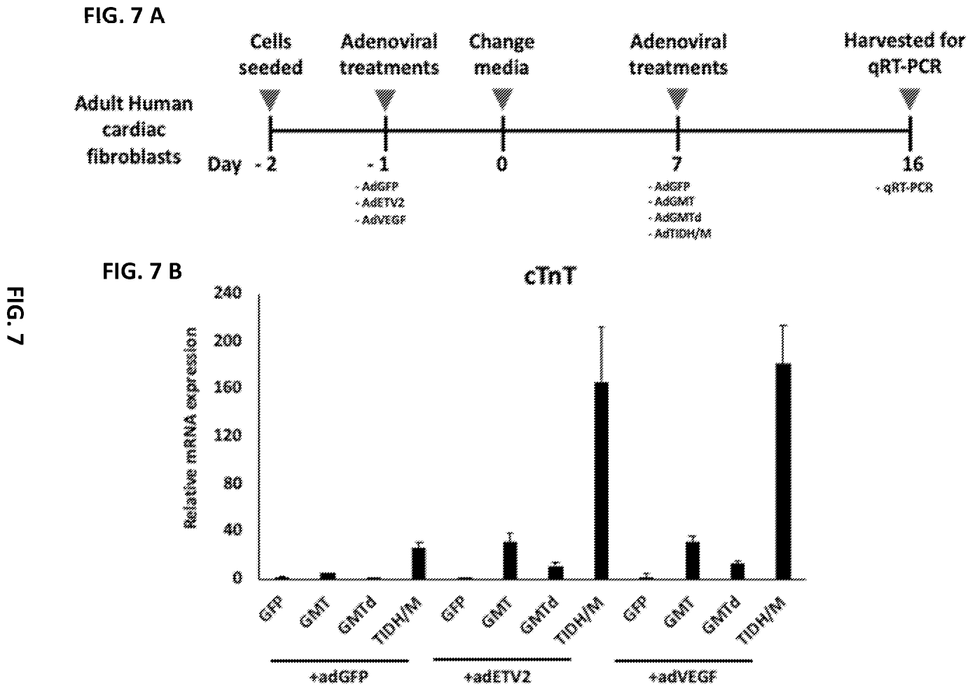

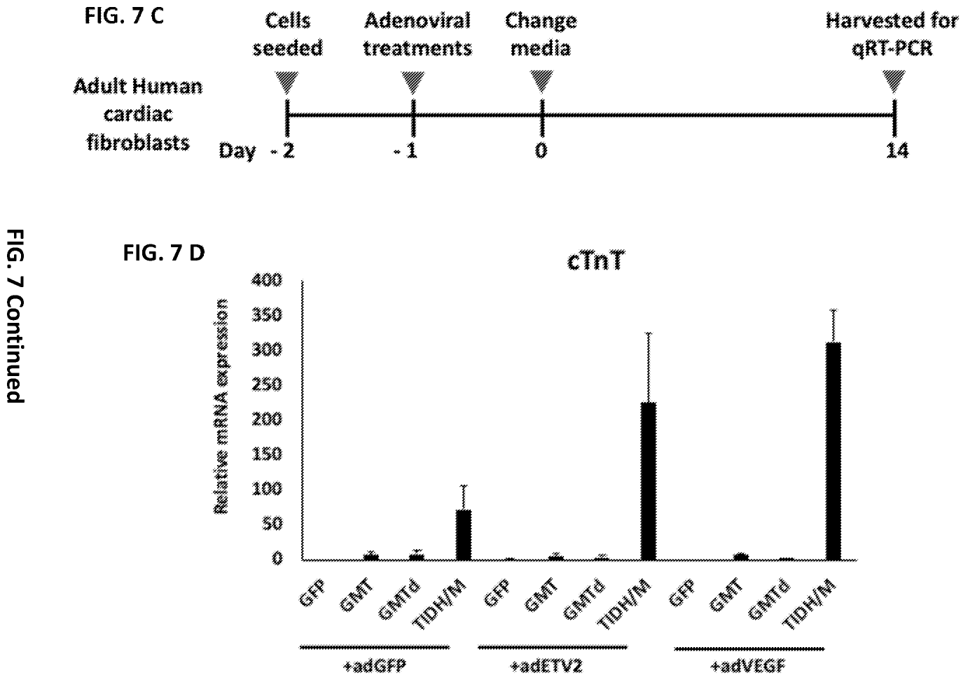

As shown in FIG. 7 A-B, human cardiac fibroblasts were treated with adenovirus encoding GFP (adGFP), ETV2 (adETV2) or VEGF (adVEGF), and seven days later cells were treated for 14 days with adenovirus encoding GFP, GMT, GMTd or TIDH/M. The results showed that cTnT marker gene expression was significantly increased in TIDH/M treatment groups when compared to controls as assessed by qRT-PCR after indicated treatments (n=3). Data was represented as fold change, and the internal control was GAPDH.

As shown in FIG. 7 C-D, human cardiac fibroblasts were treated with adenovirus encoding GFP (adGFP), ETV2 (adETV2) or VEGF (adVEGF) with or without simultaneous treatment with adenovirus encoding GFP, GMT, GMTd or TIDH/M. Two weeks later, cTnT marker gene expression was assessed by qRT-PCR after indicated treatments (n=3), the results showed that cTnT marker gene expression was significantly increased in TIDH/M treatment groups when compared to controls. Data was represented as fold change, and the internal control was GAPDH.

Together these results showed that simultaneous and/or delayed administration of TIDH/M with ETV2 or VEGF markedly improved myocardial reprogramming.

### REFERENCES

The following references, to the extent that they provide exemplary procedural or other details supplementary to those set forth herein, are specifically incorporated herein by reference.
1. Roger, V. L. Epidemiology of heart failure. Circ. Res. 128, 1421-1434. https://doi.org/10.1161/CIRCRESAHA.121.318172 (2021).
2. Shinde, A. V. & Frangogiannis, N. G. Fibroblasts in myocardial infarction: A role in inflammation and repair. J. Mol. Cell Cardiol. 70, 74-82. https://doi.org/10.1016/j.yjmcc.2013.11.015 (2014).
3. Giacomelli, E., Mummery, C. L. & Bellin, M. Human heart disease: Lessons from human pluripotent stem cell-derived cardiomyocytes. Cell Mol. Life Sc. 74, 3711-3739. https://doi.org/10.1007/s00018-017-2546-5 (2017).
4. Ieda, M. et al. Direct reprogramming of fibroblasts into functional cardiomyocytes by defined factors. Cell 142, 375-386. https://doi.org/10.1016/j.cell.2010.07.002 (2010).
5. Nam, Y. J. et al. Reprogramming of human fibroblasts toward a cardiac fate. Proc. Natl. Acad. Sci. U.S.A. 110, 5588-5593. https://doi.org/10.1073/pnas.1301019110 (2013).
6. Qian, L. et al. In vivo reprogramming of murine cardiac fibroblasts into induced cardiomyocytes. Nature 485, 593-598. https://doi.org/10.1038/nature11044 (2012).
7. Song, K. et al. Heart repair by reprogramming non-myocytes with cardiac transcription factors. Nature 485, 599-604. https://doi.org/10.1038/nature11139 (2012).
8. Patel, V. et al. p63 Silencing induces reprogramming of cardiac fibroblasts into cardiomyocyte-like cells. J. Thorac. Cardiovasc. Surg. 156, 556-565. https://doi.org/10.1016/j.jtcvs.2018.03.162 (2018).
9. Singh, V. P. et al. MiR-590 promotes transdifferentiation of porcine and human fibroblasts toward a cardiomyocyte-like fate by directly repressing specificity protein 1. J. Am. HeartAssoc. https://doi.org/10.1161/jaha.116.003922 (2016).
10. Singh, V. P. et al. Enhanced generation of induced cardiomyocytes using a small-molecule cocktail to overcome barriers to cardiac cellular reprogramming. J. Am. Heart Assoc. 9, e015686. https://doi.org/10.1161/jaha.119.015686 (2020).
11. Wang, L. et al. Down-regulation of Beclin1 promotes direct cardiac reprogramming. Sci. Transl. Med. https://doi.org/10.1126/scitranslmed.aay7856 (2020).
12. Abad, M. et al. Notch inhibition enhances cardiac reprogramming by increasing MEF2C transcriptional activity. Stem Cell Rep. 8, 548-560. https://doi.org/10.1016/j.stemcr.2017.01.025 (2017).
13. Cao, N. et al. Conversion of human fibroblasts into functional cardiomyocytes by small molecules. Science 352, 1216-1220. https://doi.org/10.1126/science.aaf1502 (2016).
14. Christoforou, N. et al. Core transcription factors, microRNAs, and small molecules drive transdifferentiation of human fibroblasts towards the cardiac cell lineage. Sci. Rep. 7, 40285. https://doi.org/10.1038/srep40285 (2017).
15. Ebrahimi, B. Reprogramming barriers and enhancers: Strategies to enhance the efficiency and kinetics of induced pluripotency. Cell Regen. 4, 10. https://doi.org/10.1186/s13619-015-0024-9 (2015).
16. Vaseghi, H., Liu, J. & Qian, L. Molecular barriers to direct cardiac reprogramming. Protein Cell 8, 724-734. https://doi.org/10.1007/s13238-017-0402-x (2017).
17. Talkhabi, M., Zonooz, E. R. & Baharvand, H. Boosters and barriers for direct cardiac reprogramming. Life Sci. 178, 70-86. https://doi.org/10.1016/j.lfs.2017.04.013 (2017).
18. Kawamura, T. et al. Linking the p53 tumour suppressor pathway to somatic cell reprogramming. Nature 460, 1140-1144. https://doi.org/10.1038/nature08311 (2009).
19. Bao, X. et al. The p53-induced lincRNA-p21 derails somatic cell reprogramming by sustaining H3K9me3 and CpG methylation at pluripotency gene promoters. Cell Res. 25, 80-92. https://doi.org/10.1038/cr.2014.165 (2015).
20. Hong, H. et al. Suppression of induced pluripotent stem cell generation by the p53-p21 pathway. Nature 460, 1132-1135. https://doi.org/10.1038/nature08235 (2009).
21. Itahana, K. et al. Control of the replicative life span of human fibroblasts by p16 and the polycomb protein Bmi-1. Mol. Cell Biol. 23, 389-401. https://doi.org/10.1128/mcb.23.1.389-401.2003 (2003).
22. Chakravarti, D. et al. Induced multipotency in adult keratinocytes through down-regulation of A Np63 or DGCR8. Proc. Natl. Acad. Sci. U.S.A. 111, E572-E581. https://doi.org/10.1073/pnas.1319743111 (2014).
23. Rasmussen, M. A. et al. Transient p53 suppression increases reprogramming of human fibroblasts without affecting apoptosis and DNA damage. Stem Cell Rep. 3, 404-413. https://doi.org/10.1016/j.stemcr.2014.07.006 (2014).
24. Flores, E. R. The roles of p63 in cancer. Cell Cycle 6, 300-304. https://doi.org/10.4161/cc.6.3.3793 (2007).
25. Venkatanarayan, A., Raulji, P., Norton, W. & Flores, E. R. Novel therapeutic interventions for p53-altered tumors through manipulation of its family members, p63 and p73. Cell Cycle 15, 164-171. https://doi.org/10.1080/15384101.2015.1121333 (2016).
26. Ramsey, M. R., He, L., Forster, N., Ory, B. & Ellisen, L. W. Physical association of HDAC1 and HDAC2 with p63 mediates transcriptional repression and tumor maintenance in squamous cell carcinoma. Cancer Res. 71, 4373-4379. https://doi.org/10.1158/0008-5472.Can-11-0046 (2011).
27. Mathison, M. et al. Cardiac reprogramming factor Gata4 reduces postinfarct cardiac fibrosis through direct repression of the profibrotic mediator snail. J. Thorac. Cardiovasc. Sury. 154, 1601-1610. https://doi.org/10.1016/j.jtcvs.2017.06.035 (2017).
28. Mathison, M. et al. Fibroblast transition to an endothelial “trans” state improves cell reprogramming efficiency. Sc. Rep. 11, 22605. https://doi.org/10.1038/s41598-021-02056-x (2021).
29. Patel, V., Mathison, M., Singh, V. P., Yang, J. & Rosengart, T. K. Direct cardiac cellular reprogramming for cardiac regeneration. Curr. Treat. Opt. Cardiovasc. Med. 18, 58. https://doi.org/10.1007/si1936-016-0480-8 (2016).
30. Singh, V. P. et al. Hippo pathway effector Tead1 induces cardiac fibroblast to cardiomyocyte reprogramming. J. Am. Heart Assoc. 10, e022659 (2021).
31. Miyamoto, K. et al. Direct in vivo reprogramming with sendai virus vectors improves cardiac function after myocardial infarction. Cell Stem Cell 22, 91-103. https://doi.org/10.1016/j.stem.2017.11.010 (2018).
32. Mathison, M. et al. “Triplet” polycistronic vectors encoding Gata4, Mef2c, and Tbx5 enhances postinfarct ventricular functional improvement compared with singlet vectors. J. Thorac. Cardiovasc. Surg. 148, 1656-1664. https://doi.org/10.1016/j.jtcvs.2014.03.033 (2014).
33. Mathison, M. et al. In situ reprogramming to transdifferentiate fibroblasts into cardiomyocytes using adenoviral vectors: Implications for clinical myocardial regeneration. J. Thorac. Cardiovasc. Surg. 153, 329-339. https://doi.org/10.1016/j.jtcvs.2016.09.041 (2017).
34. Zhou, H., Dickson, M. E., Kim, M. S., Bassel-Duby, R. & Olson, E. N. Akt1/protein kinase B enhances transcriptional reprogramming of fibroblasts to functional cardiomyocytes. Proc. Natl. Acad. Sci. U.S.A. 112, 11864-11869. https://doi.org/10.1073/pnas.1516237112 (2015).
35. Fu, X., Wu, S., Li, B., Xu, Y. & Liu, J. Functions of p53 in pluripotent stem cells. Protein Cell 11, 71-78. https://doi.org/10.1007/s13238-019-00665-x (2020).
36. Lin, T. & Lin, Y. p53 switches off pluripotency on differentiation. Stem Cell Res. Ther. 8, 44. https://doi.org/10.1186/s13287-017-0498-1 (2017).
37. Candi, E. et al. Metabolic pathways regulated by p63. Biochem. Biophys. Res. Commun. 482, 440-444. https://doi.org/10.1016/j.bbrc.2016.10.094 (2017).
38. Galoczova, M., Coates, P. & Vojtesek, B. STAT3, stem cells, cancer stem cells and p63. Cell Mol. Biol. Lett. 23, 12. https://doi.org/10.1186/s11658-018-0078-0 (2018).
39. Guo, X. et al. TAp63 induces senescence and suppresses tumorigenesis in vivo. Nat. Cell Biol. 11, 1451-1457. https://doi.org/10.1038/ncb1988 (2009).
40. Ratovitski, E. A. Tumor protein p63/microRNA network in epithelial cancer cells. Curr. Genomics 14, 441-452. https://doi.org/10.2174/13892029113146660011 (2013).
41. Soares, E. & Zhou, H. Master regulatory role of p63 in epidermal development and disease. Cell Mol. Life Sci. 75, 1179-1190. https://doi.org/10.1007/s00018-017-2701-z (2018).
42. Venkatanarayan, A. et al. IAPP-driven metabolic reprogramming induces regression of p53-deficient tumours in vivo. Nature 517, 626-630. https://doi.org/10.1038/nature13910 (2015).
43. Yi, M. et al. TP63 links chromatin remodeling and enhancer reprogramming to epidermal differentiation and squamous cell carcinoma development. Cell Mol. Life Sci. 77, 4325-4346. https://doi.org/10.1007/s00018-020-03539-2 (2020).
44. Liu, Z. et al. Re-patterning of H3K27me3, H3K4me3 and DNA methylation during fibroblast conversion into induced cardiomyocytes. Stem Cell Res. 16, 507-518. https://doi.org/10.1016/j.scr.2016.02.037 (2016).
45. Kim, K. P. et al. Permissive epigenomes endow reprogramming competence to transcriptional regulators. Nat. Chem. Biol. 17, 47-56. https://doi.org/10.1038/s41589-020-0618-6 (2021).
46. Kurotsu, S. et al. Soft matrix promotes cardiac reprogramming via inhibition of YAP/TAZ and suppression of fibroblast signatures. Stem Cell Rep. 15, 612-628. https://doi.org/10.1016/j.stemcr.2020.07.022 (2020).
47. Zhao, Y. et al. High-efficiency reprogramming of fibroblasts into cardiomyocytes requires suppression of pro-fibrotic signalling. Nat. Commun. 6, 8243. https://doi.org/10.1038/ncomms9243 (2015).
48. Zhou, Y. et al. Bmil is a key epigenetic barrier to direct cardiac reprogramming. Cell Stem Cell 18, 382-395. https://doi.org/10.1016/j.stem.2016.02.003 (2016).
49. Lau, C. H. & Tin, C. The synergy between CRISPR and chemical engineering. Curr. Gene Ther. 19, 147-171. https://doi.org/10.2174/1566523219666190701100556 (2019).
50. Liu, P., Chen, M., Liu, Y., Qi, L. S. & Ding, S. CRISPR-based chromatin remodeling of the endogenous Oct4 or Sox2 locus enables reprogramming to pluripotency. Cell Stem Cell 22, 252-261. https://doi.org/10.1016/j.stem.2017.12.001 (2018).
51. Liu, Z. et al. Application of various delivery methods for CRISPR/dCas9. Mol. Biotechnol. 62, 355-363. https://doi.org/10.1007/s12033-020-00258-8 (2020).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising a recombinant protein and/or recombinant nucleic acid encoding the same, consisting of, or consisting essentially of, a p63-Transactivation Inhibitory domain (p63-TID) polypeptide or a functional derivative and/or a functional fragment thereof; wherein the p63-TID polypeptide comprises, consists essentially of, consists of, or is, a sequence that is at least or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NO: 1.

2. The composition of claim 1, wherein the p63-TID polypeptide comprises, consists of, or consists essentially of the sequence of SEQ ID NO: 1.

3. The composition of claim 1 or 2, wherein the polypeptide is comprised in a pharmaceutically acceptable carrier.

4. The composition of any one of claims 1-3, wherein the functional derivative or fragment thereof comprises 1, 2, 3, 4, 5, 6, 7, 8, 9, 10, 11, 12, 13, 14, 15, 16, 17, 18, 19, or 20 or more amino acid alterations compared to SEQ ID NO: 1.

5. The composition of any one of claims 1-4, wherein the functional derivative and/or the functional fragment thereof comprises an N-terminal truncation of SEQ ID NO: 1.

6. The composition of claim 5, wherein the truncation is no more than 1, 2, 3, 4, 5, 6, 7, 8, 9, 10, 12, 15, 20, 25, 30, 35, 40, 45, 50, 55, or 60 amino acids or wherein the truncation is at least 1, 2, 3, 4, 5, 6, 7, 8, 9, 10, 12, 15, 20, 25, 30, 35, 40, 45, 50, 55, or 60 amino acids.

7. The composition of any one of claims 1-6, wherein the functional derivative and/or the functional fragment thereof comprises a C-terminal truncation of SEQ ID NO: 1.

8. The composition of claim 7, wherein the truncation is no more than 1, 2, 3, 4, 5, 6, 7, 8, 9, 10, 12, 15, 20, 25, 30, 35, 40, 45, 50, 55, or 60 amino acids or is at least 1, 2, 3, 4, 5, 6, 7, 8, 9, 10, 12, 15, 20, 25, 30, 35, 40, 45, 50, 55, or 60 amino acids.

9. The composition of any one of claims 1-8, wherein the functional derivative and/or the functional fragment thereof comprises an internal deletion in SEQ ID NO: 1.

10. The composition of claim 9, wherein the internal deletion is no more than 1, 2, 3, 4, 5, 6, 7, 8, 9, 10, 12, 15, 20, 25, 30, 35, 40, 45, 50, 55, or 60 amino acids or is at least 1, 2, 3, 4, 5, 6, 7, 8, 9, 10, 12, 15, 20, 25, 30, 35, 40, 45, 50, 55, or 60 amino acids.

11. The composition of any one of claims 1-10, wherein the p63-TID functional derivative and/or the fragment thereof may comprise sequence that is at least or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NO: 1.

12. The composition of any one of claims 1-11, wherein the polypeptide is labeled.

13. The composition of any one of claims 1-11, wherein one or more of the amino acids comprises a post-translational modification.

14. The composition of claim 13, wherein the post-translational modification comprises phosphorylation, acetylation, ubiquitination, acylation, methylation, or a combination thereof.

15. A composition comprising one or more recombinant nucleic acid, wherein said nucleic acid encodes the polypeptide of any one of claims 1-14, or a functional derivative and/or a functional fragment thereof.

16. The composition of claim 15, wherein said nucleic acid is DNA.

17. The composition of claim 15, wherein said nucleic acid is RNA.

18. The composition of any one of claims 15-17, wherein said nucleic acid is comprised in one or more viral vector.

19. The composition of claim 18, wherein the viral vector is a lentiviral vector.

20. The composition of claim 18, wherein the viral vector is an adenoviral vector.

21. The composition of claim 18, wherein the viral vector is an adeno associated virus (AAV) viral vector.

22. The composition of any one of claims 15-17, wherein said nucleic acid is comprised in a non-viral vector.

23. The composition of any one of claims 15-22, wherein said nucleic acid is comprised in a pharmaceutically acceptable carrier.

24. A composition comprising one or more nucleic acid vectors, said vectors comprising the composition of any one of claims 1-23 and optionally comprising one or more cardiac cell reprogramming factors.

25. The composition of claim 24, wherein the vector comprising the composition of any one of claims 1-23 is the same vector that comprises one or more cardiac cell reprogramming factors.

26. The composition of claim 24, wherein the vector comprising the composition of any one of claims 1-23 is a different vector than the vector that comprises one or more cardiac cell reprogramming factors.

27. The composition of any one of claims 24-26, further comprising a vector that comprises one or more chromatin destabilizing agents.

28. The composition of claim 27, wherein the vector that comprises the composition of any one of claims 1-27 is the same vector that comprises one or more chromatin destabilizing agents.

29. The composition of claim 27, wherein the vector that comprises the composition of any one of claims 1-28 and that comprises one or more cardiac cell reprogramming factors is the same vector that comprises one or more chromatin destabilizing agents.

30. The composition of claim 27, wherein the vector that comprises the composition of any one of claims 1-27 and that comprises one or more cardiac cell reprogramming factors is a different vector than the vector that comprises one or more chromatin destabilizing agents.

31. The composition of any one of claims 1-30, wherein the composition comprises one or both of Hand2 and Myocardin encoding nucleic acids.

32. The composition of any one of claims 1-31, wherein the composition comprises a Hand2 encoding nucleic acid.

33. The composition of any one of claims 1-32, wherein the composition comprises a myocardin encoding nucleic acid.

34. The composition of any one of claims 1-33, wherein the composition comprises Hand2 and myocardin encoding nucleic acids.

35. The composition of any one of claims 1-34, comprising one or more anti-fibrotic agents.

36. The composition of any one of claims 1-35, wherein the composition comprises Hand2, myocardin, and ETV2 and/or VEGF encoding nucleic acids.

37. The composition of claim 36, wherein the ETV2 and/or VEGF are deliverable via one or more viral vectors.

38. The composition of claim 37, wherein the viral vector is a lentiviral vector, adenoviral vector, adeno-associated viral vector, or retroviral vector.

39. The composition of claim 38, wherein the viral vector is an adenoviral vector.

40. A kit comprising the composition of any one of claims 1-39, said composition housed in a suitable container.

41. A method of in vivo reprogramming of cardiac cells, comprising the step of providing a therapeutically effective amount of one or more compositions to the heart of an individual, wherein said one or more compositions comprise the composition of any one of claims 1-39.

42. A method of treating a heart condition, comprising the step of providing a therapeutically effective amount of one or more compositions to the heart of an individual, wherein said one or more compositions comprise the composition of any one of claims 1-39.

43. The method of claim 41 or 42, further comprising the step of providing to the individual an effective amount of one or more cardiac cell reprogramming factors.

44. The method of claim 43, wherein the one or more cardiac cell reprogramming factors are a polypeptide, peptide, or nucleic acid.

45. The method of claim 43 or 44, wherein the one or more cardiac cell reprogramming factors are Hand2, myocardin, Gata4, Mef2c, Tbx5, Mesoderm posterior protein 1 (Mesp1), miR-133, miR-1, Oct4, Klf4, c-myc, Sox2, Brachyury, Nkx2.5, ETS2, ESRRG, Mrtf-A, MyoD, ZFPM2, or a combination thereof.

46. The method of any one of claims 43-45, wherein the one or more cardiac cell reprogramming factors are one or both of Hand2 and myocardin nucleic acids or polypeptides.

47. The method of claim 46, wherein the one or both of Hand2 and myocardin nucleic acids and/or polypeptides are in the same composition as the composition of any one of claims 1-39.

48. The method of claim 46, wherein the one or both of Hand2 and myocardin nucleic acids and/or polypeptides are in a different composition as the composition of any one of claims 1-39.

49. The method of any one of claims 43-48, wherein the one or more compositions comprise the composition of any one of claims 1-39 and Hand2.

50. The method of any one of claims 43-48, wherein the one or more compositions comprise the composition of any one of claims 1-39 and myocardin.

51. The method of any one of claims 43-48, wherein the one or more compositions comprise the composition of any one of claims 1-39, Hand2, and myocardin.

52. The method of any one of claims 43-51, wherein the one or more compositions comprise the composition of any one of claims 1-39, Hand2, and myocardin, and VEGF and/or ETV2.

53. The method of any one of claims 43-52, wherein the composition of any one of claims 1-39 is provided before the one or more cardiac cell reprogramming factors.

54. The method of any one of claims 43-52, wherein the composition of any one of claims 1-39 is provided after the one or more cardiac cell reprogramming factors.

55. The method of any one of claims 43-54, wherein an effective amount of VEGF and/or ETV2 is provided to the individual, before or simultaneously with any one of the compositions of claims 1-39.

56. The method of any one of claims 43-55, wherein an effective amount of one or more chromatin destabilizing agents is provided to the individual.

57. The method of claim 56, wherein the one or more chromatin destabilizing agents are selected from the group consisting of Oct4, DZNep, Sall4, SOX2, KLF4, MYC, SB431542, PD0325901, Parnate, CHIR99021, A-83-01, NaB, PS48, Forskolin (FSK), 2-methyl-5-hydroxytryptamine (2-Me-5HT), D4476, VPA,CHIR99021 (CHIR), 616452, Tranylcypromine, Prostaglandin E2, Rolipram, 3-deazaneplanocin A (DZNep), 5-Azacytidine, sodium butyrate, RG108, and a combination thereof.

58. The method of claim 56 or 57, wherein the one or more chromatin destabilizing agents are provided to the individual prior to when the composition of any one of claims 1-39 is provided to the individual.

59. The method of claim 56 or 57, wherein the one or more chromatin destabilizing agents are provided to the individual prior to when the composition of any one of claims 1-39 is provided to the individual, and wherein the composition of any one of claims 1-39 is provided to the individual prior to when the one or more cardiac cell reprogramming factors are provided to the individual.

60. The method of any one of claims 41-59, wherein the cardiac cells are fibroblasts, endothelial cells, myoblasts, progenitor cells, stem cells, or a combination thereof.

61. The method of any one of claims 41-60, wherein the composition of any one of claims 1-39 comprises a nucleic acid and said nucleic acid is comprised on one or more vectors.

62. The method of any one of claims 43-61, wherein the one or more cardiac cell reprogramming factors comprise a nucleic acid and said nucleic acid is comprised on one or more vectors.

63. The method of any one of claims 56-62, wherein the one or more chromatin destabilizing agents comprise a nucleic acid and said nucleic acid is comprised on one or more vectors.

64. The method of claim 61-63, wherein said nucleic acid of the composition of any one of claims 1-39, said nucleic acid of the cardiac cell reprogramming factors, and said nucleic acid of the chromatin destabilizing agents are comprised on separate vectors.

65. The method of claim 61-63, wherein said nucleic acid of the composition of any one of claims 1-39, said nucleic acid of the cardiac cell reprogramming factors, and said nucleic acid of the chromatin destabilizing agents are comprised on the same vector.

66. The method of claim 64 or 65, wherein the vector is a viral vector or a non-viral vector.

67. The method of claim 66, wherein the non-viral vector is a nanoparticle, plasmid, liposome, or a combination thereof.

68. The method of claim 66, wherein the viral vector is an adenoviral, lentiviral, retroviral, or adeno-associated viral vector.

69. The method of claim 68, wherein the viral vector is at a multiplicity of infection (MOI) of about 20, about 50, or about 100.

70. The method of claim 69, wherein the viral vector is at a MOI of about 50.

71. The method of any one of claims 66-70, wherein the composition of any one of claims 1-39, Hand2, and/or myocardin nucleic acids are comprised on a lentiviral vector.

72. The method of any one of claims 60-70, wherein the composition of any one of claims 1-39, Hand2, and/or myocardin nucleic acids are comprised on an adenoviral vector.

73. The method of any one of claims 41-59, wherein the one or more cardiac cell reprogramming factors comprise a nucleic acid and said nucleic acid is a DNA or RNA molecule.

74. The method of any one of claims 56-59, wherein the one or more chromatin destabilizing agents comprise a nucleic acid and said nucleic acid is a DNA or RNA molecule.

75. The method of any one of claims 41-74, further comprising the step of delivering to the individual an additional cardiac therapy.

76. The method of claim 75, wherein the additional cardiac therapy comprises drug therapy, surgery, ventricular assist device (VAD) implantation, video assisted thoracotomy (VAT) coronary bypass, percutaneous coronary intervention (PCI), or a combination thereof.

77. The method of any one of claims 41-76, wherein the cardiac cell is a dividing cell or a non-dividing cell.

78. The method of any one of claims 61-74, wherein a promoter on the vector is a cell-specific promoter.

79. The method of any one of claims 61-74, wherein a promoter on the vector is a fibroblast-specific promoter.

80. The method of any one of claims 41-79, wherein the providing step is further defined as injecting the composition of any one of claims 1-39 into the heart.

81. The method of any one of claims 43-80, wherein if the one or more composition comprises one or more cardiac cell reprogramming factors, the providing step is further defined as injecting the one or more cardiac cell reprogramming factors into the heart.

82. The method of any one of claims 43-81, wherein if the one or more composition comprises one or more chromatin stabilizing agents, the providing step is further defined as injecting the one or more chromatin stabilizing agents into the heart.

83. The method of any one of claims 41-82, further comprising the step of providing one or more anti-fibrotic agents.

84. The method of claim 83, wherein the one or more anti-fibrotic agents comprise at least one anti-Snail agent.

85. The method of claim 84, wherein the anti-Snail agent is a siRNA, shRNA, antibody, or small molecule.

86. The method of any one of claims 41-85, wherein the one or more composition is delivered to one or more areas of myocardial scar tissue.

87. The method of any one of claims 41-85, wherein the one or more composition is delivered to one or more areas that is not myocardial scar tissue.

88. The method of any one of claims 41-85, wherein the one or more composition is delivered globally to the heart.

89. The method of any one of claims 41-88, wherein when the one or more composition localizes to scar cells.

90. The method of claim 89, wherein the scar cells are fibroblasts cells.

91. The method of claim 89 or 90, wherein the cells form sarcomeres.

92. The method of any one of claims 89-91, wherein the cells can contract.

93. The method of any one of claims 89-92, wherein the one or more composition comprises nucleic acid comprising a fibroblast-specific promoter.

94. The method of claim 93, wherein the fibroblast-specific promoter is periostin.

95. The method of any one of claims 89-94, wherein the delivery is directly to the heart.

96. The method of any one of claims 89-95, wherein the delivery is localized using a cardiac-specific vector.

97. The method of claim 96, wherein the cardiac-specific vector is AAV(9).

98. A method of treating a heart condition, comprising: the step of providing a therapeutically effective amount of one or more compositions to the heart of an individual, wherein said one or more compositions comprises, consist essentially of, or consist of:
A) a p63-Transactivation Inhibitory domain (p63-TID) polypeptide and/or a functional derivative and/or a functional fragment thereof, and/or a nucleotide encoding the same; wherein the p63-TID polypeptide comprises, consists essentially of, consists of, or is, a sequence that is at least or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to SEQ ID NO: 1;
B) a Hand2 polypeptide and/or a functional derivative and/or a functional fragment thereof, and/or a nucleotide encoding the same;
C) a myocardin polypeptide and/or a functional derivative and/or a functional fragment thereof, and/or a nucleotide encoding the same;
D) an ETV2 polypeptide and/or a functional derivative and/or a functional fragment thereof, and/or a nucleotide encoding the same; and/or
E) a VEGF polypeptide and/or a functional derivative and/or a functional fragment thereof, and/or a nucleotide encoding the same.

99. The method of claim 98, comprising, consisting essentially of, or consisting of providing A, B, C, and D.

100. The method of claim 98, comprising, consisting essentially of, or consisting of providing A, B, C, and E.

101. The method of any one of claims 98-100, wherein D and/or E are provided on the same day as A, B, and C.

102. The method of any one of claims 98-101, wherein D and/or E are provided simultaneously with A, B, and C.

103. The method of any one of claims 98-100, wherein D and/or E are provided before A, B, and C.

104. The method of any one of claims 98-100 or 103, wherein D and/or E are provided at least or exactly 1, 2, 3, 4, 5, 6, 7, 8, 9, 10, 11, 12, 13, 14, 15, 16, 17, 18, 19, 20, 21, 22, 23, 24, 25, 26, 27, or 28 days, or any range derivable therein, before A, B, and C.

105. The method of any one of claims 98-100, wherein A, B, and C are provided before D and/or E.

106. The method of any one of claims 98-100 or 105, wherein A, B, and C are provided at least or exactly 1, 2, 3, 4, 5, 6, 7, 8, 9, 10, 11, 12, 13, 14, 15, 16, 17, 18, 19, 20, 21, 22, 23, 24, 25, 26, 27, or 28 days, or any range derivable therein, before D and/or E.

107. The method of any one of claims 98-106, wherein A, B, C, D, and/or E are provided in a nanoparticle, plasmid, liposome, viral vector, or any combination thereof.

108. The method of any one of claims 98-107, wherein A, B, C, D, and/or E are provided in a viral vector, wherein the viral vector is an adenoviral, lentiviral, retroviral, or adeno-associated viral vector.

109. The method of claim 108, wherein the viral vector is an adenoviral vector.
